SEQUENCE LISTING (4 sequences)

SEQ ID NO: 1 - DNA, 30 bp, artificial sequence
  Synthetic oligonucleotide primer
ctggctcgagctctagcgatgaagatacag

SEQ ID NO: 2 - DNA, 30 bp, artificial sequence
  Synthetic oligonucleotide primer
gctgctcgagcacttgcttgatccaaatcc

SEQ ID NO: 3 - DNA, 56 bp, artificial sequence
  Synthetic oligonucleotide primer
gctgggcgtggtgcctaaaaatgtctttcagtagcaagctgattgccagtttaaac

SEQ ID NO: 4 - DNA, 61 bp, artificial sequence
  Synthetic oligonucleotide primer
tcgagtttaaactggcaatcagcttgctactgaaagacatttttaggcaccacgcccagct

(19) United States
(12) Patent Application Publication
(10) Pub. No.: US 2015/0259387 A1
(43) Pub. Date: Sep. 17, 2015

(54) PRODUCTION OF A HCMV BASED VACCINE IN HUMAN AMNIOCYTE CELL LINES

(71) Applicant: CEVEC PHARMACEUTICALS GMBH, Köln (DE)

(72) Inventors: Gudrun Schiedner, Koln (DE); Bodo Plachter, Worrstadt (DE)

(21) Appl. No.: 14/436,125

(22) PCT Filed: Oct. 18, 2013
(86) PCT No.: PCT/EP2013/071887

§ 371 Date: Apr. 16, 2015

Related U.S. Application Data
(60) Provisional application No. 61/716,266, filed on Oct. 19, 2012.

(30) Foreign Application Priority Data
Oct. 19, 2012 (EP) 12189346.5
May 28, 2013 (EP) 13169567.8

(51) Int. Cl. C07K 14/005; G01N 33/569; C12N 7/00

(52) U.S. Cl. CPC C07K 14/005; C12N 7/00; G01N 33/56994; A61K 39/00

(57) ABSTRACT

The present invention relates to a method for the production of human Cytomegalovirus (HCMV) particles, the method including the steps of: (a) contacting and thereby infecting a permanent human amniocyte cell with HCMV, (b) incubating the amniocyte cell, (c) allowing expression of HCMV particles, and (d) isolating of the HCMV particles, wherein the permanent human amniocyte cell expresses the adenoviral gene products E1A and E1B and wherein the amniocyte cells are cultured in serum free medium. Furthermore, the present invention relates to HCMV particles produced by the method of the present invention as well as to a HCMV based vaccine comprising the HCMV particles, the use of the HCMV particles for use in the preparation of a HCMV based vaccine and the HCMV particles for use in the preparation of a therapeutic or diagnostic agent for the prevention or treatment of a HCMV related disease.

The present invention relates to a method for the production of human Cytomegalovirus (HCMV) particles, the method including the steps of: (a) contacting and thereby infecting a permanent human amniocyte cell with HCMV, (b) incubating the amniocyte cell, (c) allowing expression of HCMV particles, and (d) isolating of the HCMV particles, wherein the permanent human amniocyte cell expresses the adenoviral gene products E1A and E1B and wherein the amniocyte cells are cultured in serum free medium. Furthermore, the present invention relates to HCMV particles produced by the method of the present invention as well as to a HCMV based vaccine comprising the HCMV particles, the use of the HCMV particles for use in the preparation of a HCMV based vaccine and the HCMV particles for use in the preparation of a therapeutic or diagnostic agent for the prevention or treatment of a HCMV related disease.

Vaccination represents one of the most important means in the health care system for the prevention of diseases. The success of the use of vaccine components is in particular dependent on the sufficient amount of vaccination material, for example attenuated viruses that should derive from stable and easily manageable sources.

In view of safety aspects, inactivated vaccines are advantageous since the components of such inactivated vaccines are non-infectious. In this context, subviral particles have been shown to be useful for vaccination. Distinct forms of such subviral particles are so called “Dense Bodies” (DB) which represent defective viral particles which are released during infection. Such DBs allow the use in form of an inactivated vaccine which is suitable to provoke favourable immune responses (5, 6—in the list of references). DBs of HCMV are described in EP 1 159 405.

DBs are released from cells that are infected with HCMV (2). Their size varies from 130-350 nm (8). The inner protein structure is mainly composed of tegument proteins, pp65 (pUL83) and pUL25 being the most abundant constituents (10). The outer layer is made up by a lipid bilayer, derived from cellular membranes, in which viral glycoproteins are inserted (10). Besides abundant proteins, a number of further, less abundant polypeptides are contained in these particles. By virtue of the viral surface glycoproteins, DBs appear to enter fibroblasts by membrane fusion, comparable to virions (9).

Infection of healthy individuals with HCMV remains asymptomatic in most instances. Severe and life-threatening manifestations, however, may occur in immunocompromised patients, such as transplant recipients or individuals living with AIDS. Under such conditions, HCMV can infect multiple organs including lung, liver, gut, and salivary glands. HCMV is also one component of the “TORCH” complex which includes Toxoplasma gondii, Rubella virus, HCMV and Herpes simplex virus. Accordingly, HCMV can lead to congenital abnormalities in newborns and toddlers, following primary infection or, less likely, reactivation of HCMV during pregnancy. Sensorineural hearing loss, vision impairment and various degrees of mental disabilities are the most momentous manifestations in this setting. HCMV is a member of the Beta-herpesviridae family. These viruses are characterized by their strict species-specificity and their slow replication in cell culture. The genome of HCMV consists of a double-stranded DNA genome of about 230,000 base pairs, encoding a set of approximately 165 genes. The infectious HCMV-virions of about 200 nm in diameter are composed of an icosahedral capsid, containing the genome and a tegument layer. The tegument proteins define the matrix between the capsid and the outer viral envelope that consists of a lipid bilayer derived from cellular membranes. Viral glycoproteins, inserted into this matrix, are engaged in adsorption and penetration of host cells.

Viral tegument proteins, in particular pp65, have been identified as major target antigens of the T lymphocyte response against HCMV (11). Moreover, neutralizing antibodies are considered to be major effectors to prevent infection. Such antibodies are directed against HCMV surface glycoproteins, namely glycoprotein B (gB, gpUL55) or glycoprotein H (gH, gpUL75). pp65, gB, and gH are integral constituents of DB.

HCMV particles, such as DBs, are currently produced according to the prior art using human fibroblast cell cultures. The use of fibroblasts is however very complex. The use of fibroblast cell culture for clinical grade vaccine production is limited by lack of ready access to GMP-compliant fibroblast cells. In order to be approved by regulatory agencies, production cells need to be free of any infectious proteins and agents and their entire history must be documented. Usually fibroblast cells are non-transformed primary cells isolated from tissue. Among fibroblast cells, MRC-5 cells are best characterized for clinical production of vaccines and are derived from normal lung tissue of a 14-week-old male fetus isolated in 1966. MRC-5 cells grow as adherent cells in serum-containing medium and are capable of attaining 42-46 population doublings before onset of decline in proliferation. For production of vaccines used in patents, cells from a well-characterized and certified MCB with specific population doubling levels or passage levels need to be used. However, the limited population doublings of MRC-5 cells restricts the use of these cells in manufacturing to only a very small number of passages. Even though MRC-5 cells are used already for the production of numerous vaccines, they are not easy to use in large-scale production using bioreactor technologies since they only grow as adherent cells and require the use of microcarriers or multilayer methods in serum-containing medium. In addition, they cannot be engineered in order to express complementing proteins for production of vaccine vectors that require complementation.

Furthermore, the culturing of human fibroblasts necessarily requires the addition of serum of animal origin, such as fetal calf serum, FCS, to the cell cultures. This is a disadvantage since the serum of animal origin is discussed to be a potentially harmful source which may deliver pathogens. The detection of such pathogens is however difficult or impossible. Particular concerns relate to the possible outcome of bovine spongiform encephalopathy (BSE) which is caused by misfolded proteins called prions. Further, fetal calf serum is a complex mixture containing unknown compounds and these can vary among different batches. In addition, well-known and unidentified proteins from animal source may cause allergies or side effects in patients. For production of clinical grade material, Good Manufacturing Practice (GMP) as defined by both the European Medicines Agency and the US Food and Drug Administration needs to be employed and require traceability of raw materials and consistency in batch composition. Therefore, FCS should not be used under GMP conditions, which are, however, required for the production of a safe and reliable vaccine or pharmaceutical composition. Thus, there is a demand to provide vaccine production methods which do not necessarily involve the use of potentially harmful substances.

In summary, the substitution of primary cells by a continuous serum-free cell line has numerous advantages over primary fibroblast cell culture.

Thus, there is a demand to provide vaccine production methods which do not necessarily involve the use of potentially harmful substances.

Therefore, the objective technical problem underlying the present invention is the provision of a method for the production of HCMV particles as well as the provision of a HCMV based vaccine which does not involve the use of potentially harmful substances such as animal serum.

This technical problem is solved according to the subject-matter as defined in the claims.

The invention is illustrated according to the following figures:

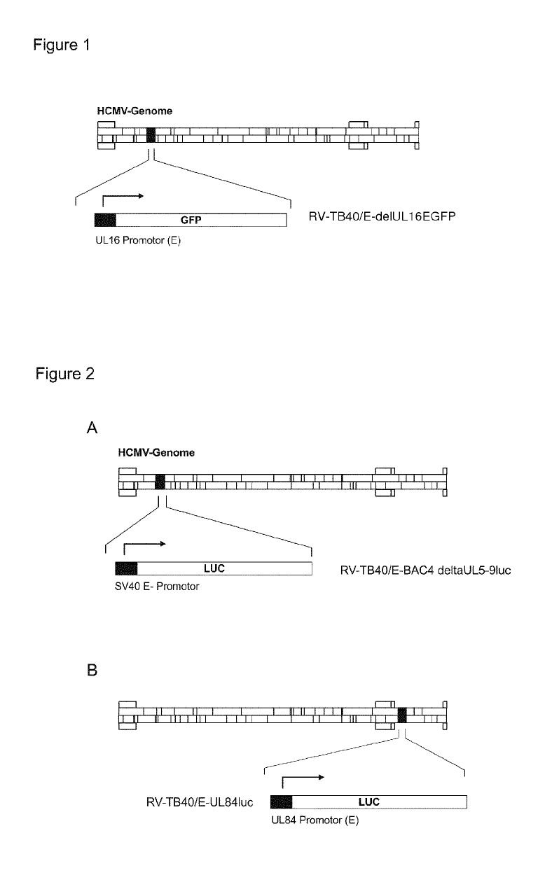

FIG. 1 shows a schematic representation of the recombinant virus RV-TB40/E-delUL16EGFP. This virus is a derivative of HCMV strain TB40/E, expressing the Green fluorescent protein (GFP) under transcriptional control of the early UL16—promotor of HCMV (3).

FIG. 2 shows a schematic representation of recombinant viruses RV-TB40/E-BAC4 deltaUL5-9luc and RV-TB40/E-UL84luc. FIG. 2A, recombinant virus RV-TB40/E-BAC4 deltaUL5-9luc expresses the firefly luciferase under the control of the SV40 early promotor. The genomic region UL5-UL9 was replaced in constructing this virus (7). FIG. 2B, recombinant virus RV-TB40/E-UL84luc (generously provided by Thomas Stamminger, Erlangen) expresses the firefly luciferase under control of the early UL84 promotor of HCMV.

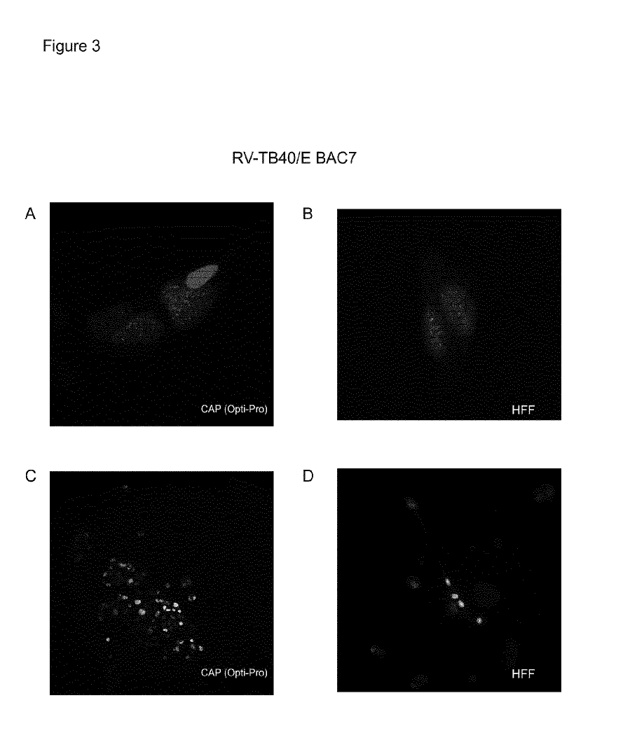

FIG. 3 shows the results of an indirect immunofluorescence (IF) analysis of adherent CAP cells and adherent human foreskin fibroblasts (HFF), infected with the BAC-derived virus RV-TB40/E BAC7 (generously provided by Christian Sinzger, Ulm) FIG. 3A and FIG. 3C show representations of infected adherent CAP cells, stained for IE1-pp72 expression (different magnifications). FIG. 3B and FIG. 3D show representations of infected HFF, stained for IE1-pp72 expression (different magnifications).

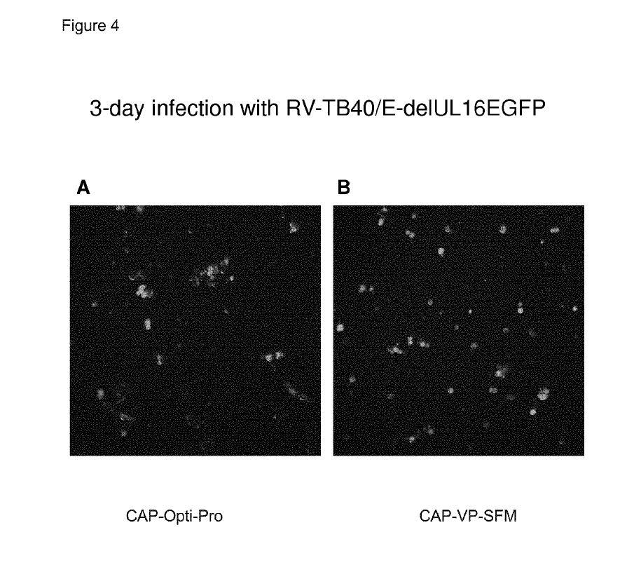

FIG. 4 shows representations of direct fluorescence analysis of CAP cells, infected with virus RV-TB40/E-delUL16EGFP. FIG. 4A shows a representation of infected adherent CAP cells cultivated in serum-containing Opti-Pro medium. FIG. 4B shows a representation of infected adherent CAP cells cultivated in VP-SFM medium in the absence of fetal calf serum.

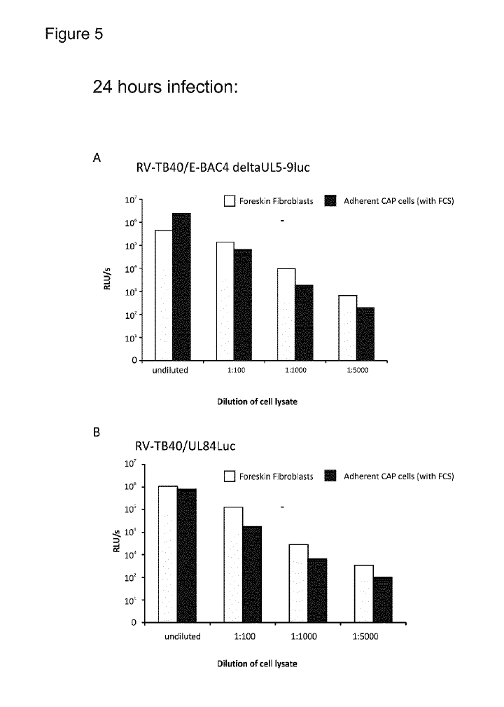

FIG. 5 shows a bar chart representing the results of the luciferase expression 24 hours post infection in adherent HFF and CAP cells (cultivated in the presence of serum) infected with HCMV expressing luciferase from different promoters. FIG. 5A shows a bar chart representing the luciferase activity, measured following a 24 hour infection with RV-TB40/E deltaUL5-9luc. FIG. 5B shows a bar chart representing the luciferase activity measured following a 24 hour infection with RV-TB40/E-UL84luc.

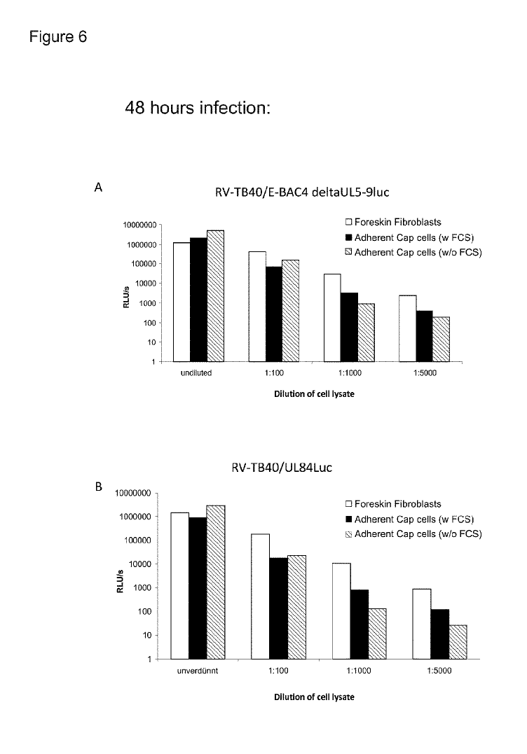

FIG. 6 shows a bar chart representing the results of the luciferase expression 48 hours post infection of adherent CAP cells, kept in the presence of FCS (w FCS) or in the absence of FCS (w/o FCS) or HFF (cultivated in the presence of FCS) and infected with HCMV expressing luciferase from different promoters. FIG. 6A, shows a bar chart representing the luciferase activity, measured following a 48-hour infection with RV-TB40/E deltaUL5-9luc. FIG. 6B shows a bar chart representing the luciferase activity, measured following a 48-hour infection with RV-TB40/E-UL84luc.

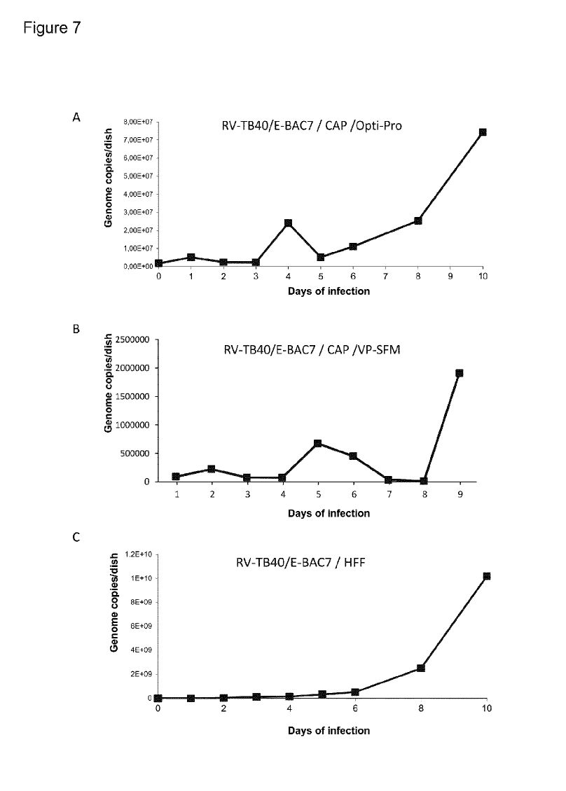

FIG. 7 shows schematically the course of the total number of viral genome copies in the dish in relation to time of infection of adherent CAP (Opti-Pro with FCS) cells, adherent CAP (VP-SFM without FCS) cells and HFF after infection, obtained by the quantitative PCR analysis. Infection was performed with HCMV strain RV-TB40/E-BAC7.

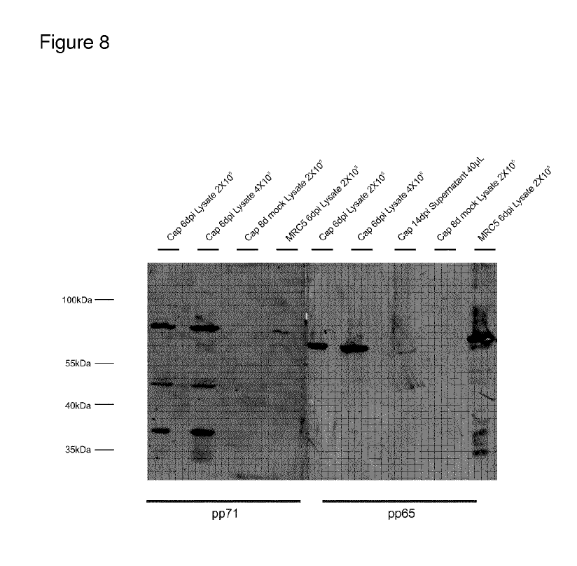

FIG. 8 shows the representation of an Odyssey® immunoblot for the expression of viral proteins following infection of CAP cells and MRC5 cells. For viral protein detection either lysate or supernatant from infected or mock-infected cells at different time points post infection and from different cell numbers are used.

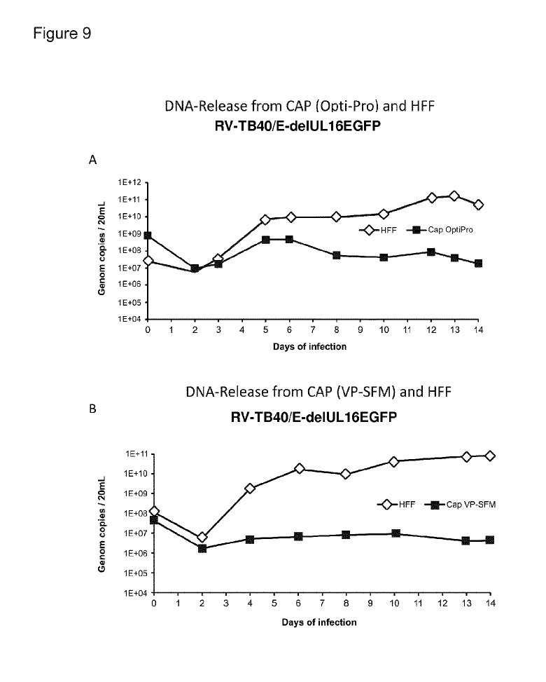

FIG. 9 schematically shows the course of the number of the released viral genomes into the cell culture supernatant of infected CAP cells cultivated in the presence of serum in Opti-Pro (FIG. 9A) or the absence of serum in VP-SFM (FIG. 9B) or infected HFF, measured by quantitative PCR analysis. Infection was performed with RV-TB40/E-delUL16EGFP.

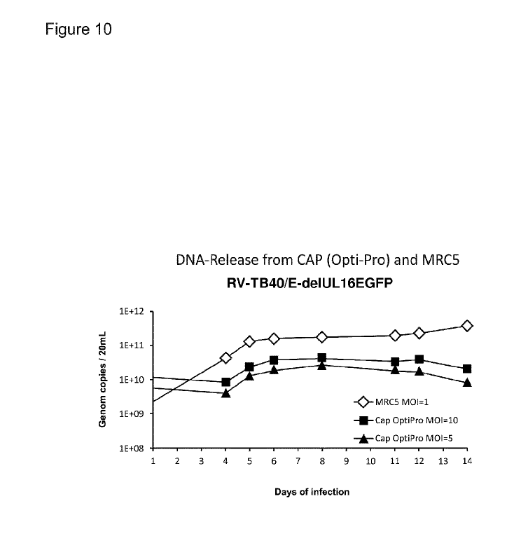

FIG. 10 schematically shows the course of the number of the released viral genomes into the cell culture supernatant of infected CAP cells or MRC5, measured by quantitative PCR analysis (with medium exchange at day 1 of infection). Infection was performed with RV-TB40/E-delUL16EGFP using different m.o.i.s. Abbreviation: MOI: multiplicity of infection.

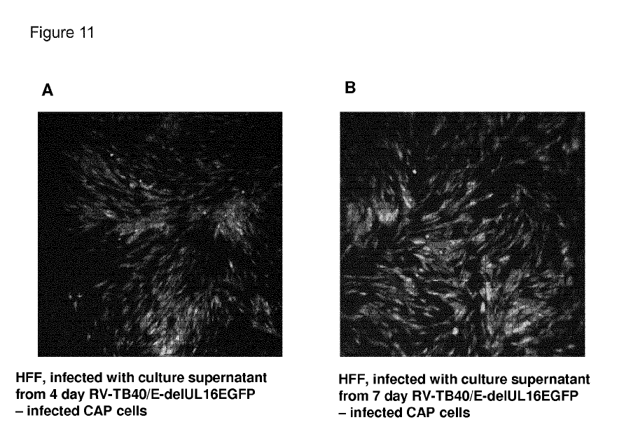

FIG. 11 shows a representation of the released infectious virus from adherent CAP cells. Fluorescence-microscopy demonstrates the direct GFP fluorescence of HFF, incubated with cell culture supernatants from 4-day infected CAP cells (A) or 7-day infected CAP cells (B). Infection was performed with HCMV strain RV-TB40/E-delUL16EGFP.

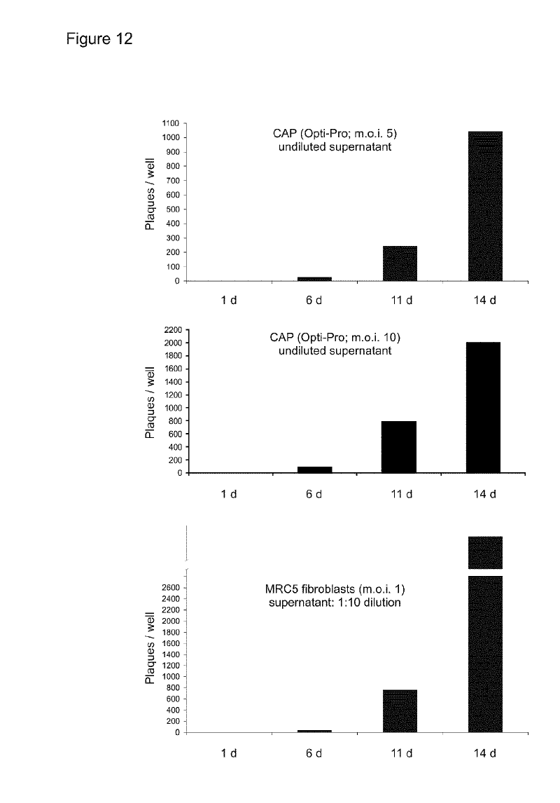

FIG. 12 shows a bar chart representing the results of plaque assays performed on HFF as quantitation for the release of infectious HCMV into the culture supernatant of infected adherent CAP or MRC5 cells. Abbreviation: m.o.i.: multiplicity of infection.

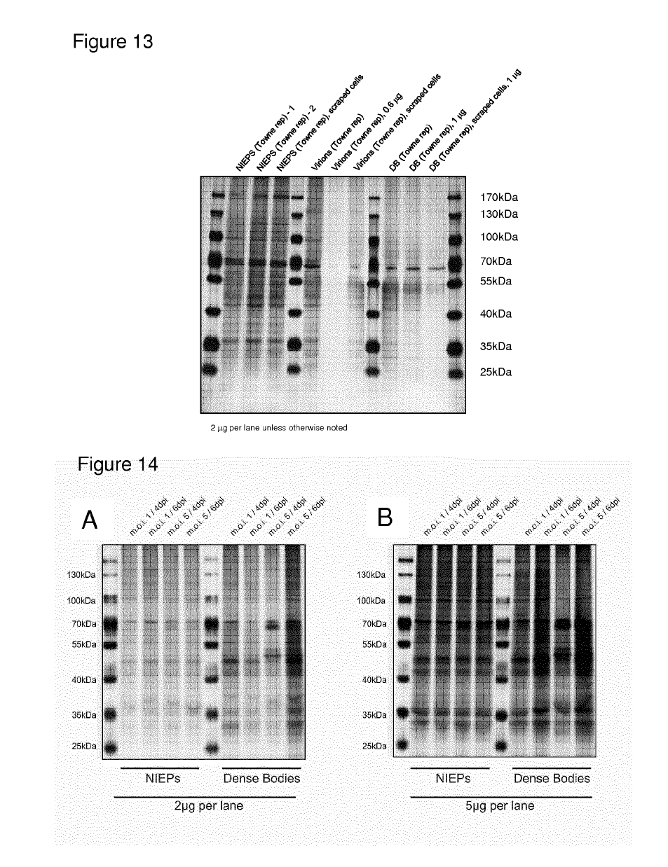

FIG. 13 shows a SDS-polyacrylamide gel followed by silver staining of proteins obtained from different particles fractions of glycerol-tartrate gradients from adherent CAP cells infected with Towneᵣₑₚ strain. Towneᵣₑₚ is a derivative of the Towne strain of human cytomegalovirus (HCMV), repaired for the expression of a functional UL130 protein. Different lanes show proteins either from non-infectious enveloped particles (NIEPs), virions or Dense Bodies (DB) released into the supernatants of infected CAP cells. The molecular masses of the proteins, used as standard are indicated. 2 μg protein of the respective fraction was separated per lane, unless otherwise noted. Abbreviation: kDa: kilodalton.

FIG. 14 shows a SDS-polyacrylamide gel followed by silver staining of proteins obtained from different particles fractions of glycerol-tartrate gradients from CAP cells cultivated in suspension in serum-free medium and infected with Towneᵣₑₚ strain (repaired for expression of functional UL130 protein). Different lanes show proteins either from non-infectious enveloped particles (NIEPs) or Dense Bodies (DB) released into the supernatants of CAP cells infected with 1 or 5 m.o.i. and infected for 4 or 6 days, respectively. Two different amounts of the materials (2 μg in FIG. 14 A and 5 μg in FIG. 14 B) were applied on the gel and separated. Abbreviations: dpi: days post infection, m.o.i: multiplicity of infection, kDa: kilodalton.

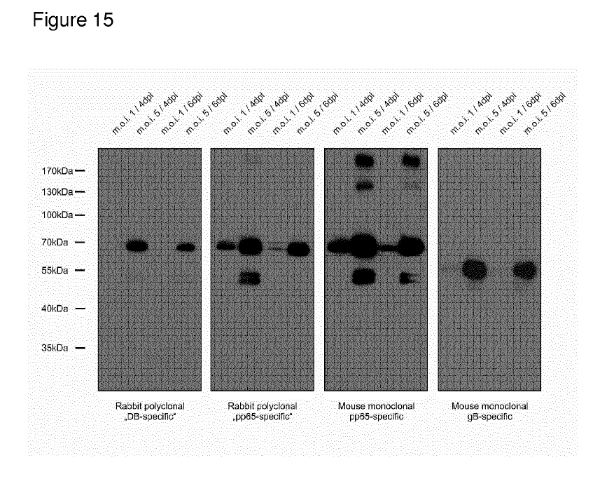

FIG. 15 shows the results of SDS-polyacrylamide gel followed by immunoblot analysis of the Dense Bodies (DB)-fractions, released from CAP cells cultivated in suspension in serum-free medium. Cells were infected with a derivative of the Towne strain of human cytomegalovirus (HCMV), repaired for the expression of a functional UL130 protein (Towneᵣₑₚ). Different polyclonal and monoclonal antibodies against pp65 and gB were used for detection. The molecular masses of the proteins, used as standard, are indicated. Abbreviations: dpi: days post infection; m.o.i: multiplicity of infection; kDa: kilodalton.

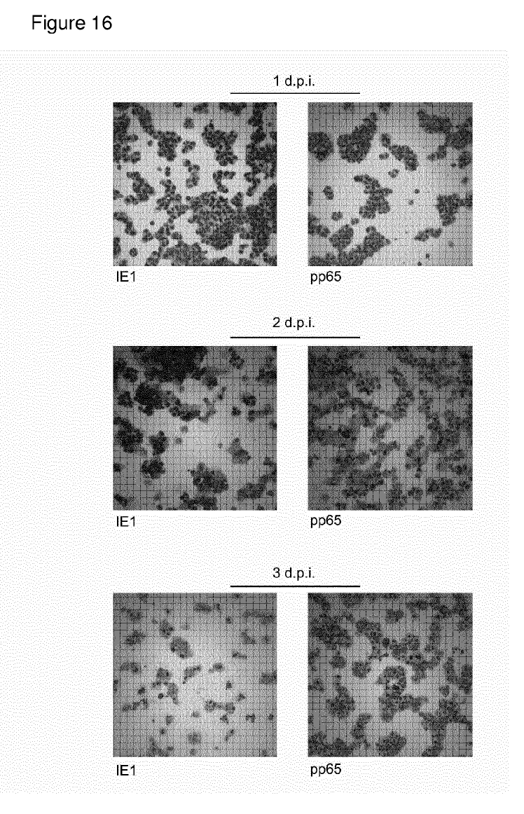

FIG. 16 shows the results from the staining of CAP cells infected with a derivative of the Towne strain of human cytomegalovirus (HCMV), repaired for the expression of a functional UL130 protein (Towneᵣₑₚ) for the nuclear IE1 protein and pp65 1, 2 and 3 days post infection. Abbreviation: dpi: days post infection.

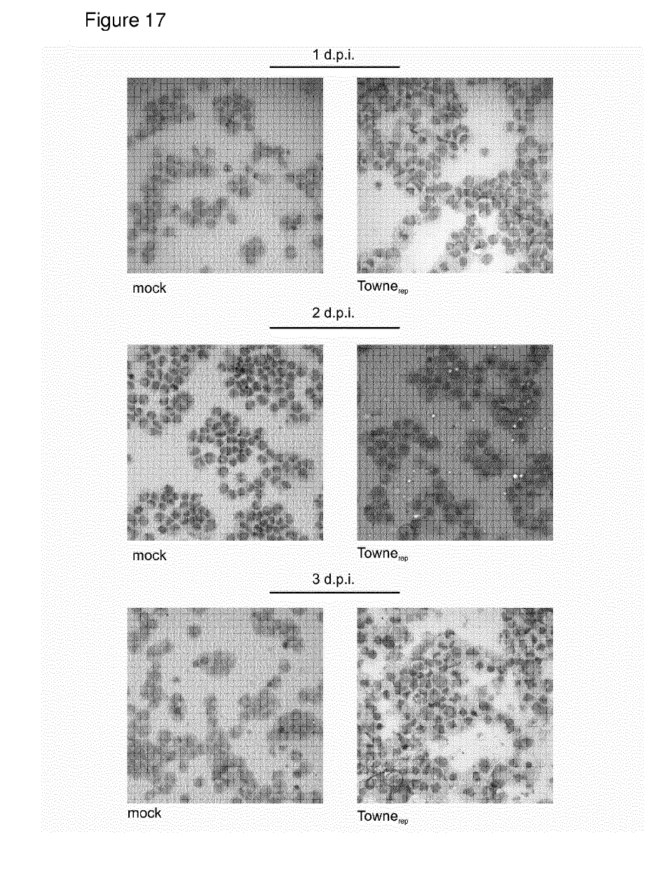

FIG. 17 shows a representation of CAP cells infected with a derivative of the Towne strain of human cytomegalovirus (HCMV), repaired for the expression of a functional UL130 protein (Towneᵣₑₚ) in comparison with control cells (mock) 1, 2 and 3 days post infection. Microscopic inspection demonstrates the cytopathic effect on the CAP cells. Abbreviation: dpi: days post infection.

The terms “human Cytomegalovirus” or “HCMV” have to be understood according to the present invention as a species of virus which belongs to the viral family of Herpesviridae. HCMV, and CMVs from other mammals than humans, are categorized in the subfamily of Beta-herpesviridae. Alternative expressions for HCMV is also human herpesvirus-5 (HHV-5).

The expression “vaccine” according to the present invention relates to a biologically or genetically generated antigen or a plurality of antigens, including proteins, protein subunits, peptides, carbohydrates, lipids, nucleic acids, inactivated or attenuated viruses, wherein the virus can be a complete virus particle or a part of a virus particle or combinations thereof. The antigen represents at least one epitope, for example a T cell and/or B cell eptiope. The antigen is recognized by immunological receptors, such as T cell receptors or B cell receptors. Accordingly, the vaccine activates, after its recognition, the immune system to target, for example, a distinct virus. An immunological response is provoked by this e.g. against viruses or viral antigens, this results in the development of antibodies and specialized T helper cells and cytolytic T cells which provide a long lasting protection. These effectors may endure between a few years and life-long, depending on the virus and depending on the respective antigen or vaccine. In the case of HCMV, viral reactivation may be suitable to boost the vaccine-induced immune response later in life. Vaccines include life and inactivated vaccines. Life vaccine comprises for example attenuated, but replication competent viruses, which are apathogenic. In the case of inactivated vaccines, viruses are killed or the vaccine only includes parts of the virus, such as antigens. The inactivation—killing—of viruses can be conducted with chemical substances, for example formaldehyde, beta-propiolactone and psoralene. The viral envelope is preserved in the course of that treatment. Furthermore, toxoid vaccines are existing which only contain the biologically inactive component—toxoid—of the toxin of an agent, for example the tetanus toxoid. The toxoid is also considered as an inactivated vaccine. Inactivated vaccines may also be composed of subfragments of viral envelope proteins. The destruction or cleavage of the virus envelope can be conducted by using detergents or strong organic solvents. Finally, inactivated vaccine further include subunit vaccines which are composed of specific components of a virus.

The expression “HCMV based vaccine” according to the present invention relates to all proteins, peptides or parts thereof as well as nucleic acids coding for said proteins, peptides or parts thereof of HCMV, and the HCMV particles itself, recombinant HCMV particles, including HCMV envelope proteins, subviral particles, virus-like particles (VLP), VLP complexes and/or parts thereof which can be used for immunization purposes against a HCMV infection.

The expression “HCMV particle” according to the present invention relates to all kind of HCMV particles or parts thereof including subviral particles, virus-like particles (VLP), VLP complexes and/or parts. Particularly, HCMV particles refer to Dense Bodies, non-infectious envelope particles (NIEPs) and/or virions.

The expression “adjuvant” according to the present invention relates to substances which are able to modulate the immunogenicity of an antigen. Adjuvants are in particular mineral salts, squalen mixtures, muramyl peptides, saponin derivates, preparations of the cell wall of mycobacteria, distinct emulsions, monophosphoryl-lipid-A, mycolic acid derivatives, non-ionic block-copolymer tensides, quil A, subunit of the choleratoxin B, polyphophazene and its derivatives, immunostimulatory complexes, cytokine adjuvants, MF59 adjuvants, mucosal adjuvants, distinct bacteria exotoxines, distinct oligonucleotides and PLG.

The expression “amniocyte” according to the present invention relates to all cells, which are present in the amniotic fluid of human origin and can be harvested via amniocentesis. These cells are derived from the amnion or from fetal tissue, which is in contact with the amniotic fluid. Three main classes of amniocyte cells are described which can be differentiated on the basis of morphological characteristics: fibroblast-like cells (F-cells), epitheloide cells (E-cells) and amniotic fluid cells (AF-cells)(4). AF-cells represent the dominant cell type.

The expression “permanent cells” or “permanent cell lines” according to the present invention relates to cells which are genetically altered such that a continuous growth in cell culture is possible under suitable culture conditions. These cells are also called immortalized cells.

The expression “primary cells” according to the present invention relates to cells which have been provided via direct removal from an organism or a tissue, and the cells are subsequently cultured. Primary cells posses only a limited life time.

The expression “adherent cell” according to the present invention relates to cells which only replicate when attached to surfaces either of microcarriers or to cell culture dishes. Usually, the attachment and the replication of adherent cells occurs in the presence of serum. Adherent cells first need to be detached from the surface in order to be used to seed new cultures.

The expression “suspension cell” according to the present invention relates to cells which can be cultivated in suspension without being attached to surfaces. Usually, the cultivation of the suspension cells can be conducted in the absence of serum. Suspension cultures can easily be passaged without the need of detaching agents.

The expression “transfection” according to the present invention relates to any method which is suitable to deliver a distinct nucleic acid or nucleic acids into cells. For example transfection can be conducted using calcium phosphate, electroporation, liposomal systems or any kind of combinations of such procedures.

The expression “CAP” or “CAP cells” according to the present invention relates to permanent human amniocyte cell lines which have been generated via immortalization of primary human amniocytes with adenoviral gene functions E1A and E1B.

The expression “CAP-T” cells according to the present invention relates to CAP cells which have been additionally stably transfected with a nucleic acid molecule including the sequence of SV40 large T antigen.

The first subject-matter of the present invention relates to a method for the production of HCMV particles, the method including the steps of: (a) contacting and thereby infecting a permanent human amniocyte cell with HCMV, (b) incubating the amniocyte cell, (c) allowing expression of HCMV particles, and (d) isolating the HCMV particles, wherein the permanent human amniocyte cell expresses the adenoviral gene products E1A and E1B.

In a preferred embodiment the HCMV particles isolated in step d) according to the method of the present invention are Dense Bodies.

After infection of the cell with HCMV, HCMV replicates inside the cell and viral proteins are expressed. The expressed proteins are then assembled into viral particles. The assembled HCMV particles may be localized intracellularly in the intracellular space of the amniocyte cell or may be released from the cell into the medium. According to the present invention, step (c) allowing the expression of HCMV particles involves the replication of HCMV DNA, HCMV protein expression and assembly of HCMV particles and release of HCMV particles from the cell.

In a preferred embodiment the amniocyte cell in step a) of the method according to the present invention is infected with a derivative of the Towne strain of human cytomegalovirus (HCMV), repaired for the expression of a functional UL130 protein (Towneᵣₑₚ) or any other strain repaired for expression of a functional UL130 protein.

In a preferred embodiment of the present invention, the amniocyte cells in step (a) are cultured in serum free medium.

In a further preferred embodiment of the present invention, the amniocyte cells in step (a) are cultured in serum-containing medium.

In a preferred embodiment of the present invention, the amniocyte cells are adherent cells or suspension cells.

In a further preferred embodiment of the present invention the amniocyte cells are suspension cells and are cultured in step a) at a density of 5×10⁴ to 5×10⁷ cells/ml in tissue culture flasks.

The culturing of the amniocyte cells in a medium without the necessity to add serum, in particular serum deriving from animal origin such as fetal calf serum—FCS, is advantageous. Serum with animal origin is potentially harmful since pathogens may reside in the serum which can then lead to diseases. Since such pathogens are difficult to be detected, it is desirable to avoid serum as addition in the culture medium to prevent any contamination of the products produced in cell culture. Moreover, serum such as FCS includes an undetermined number of unidentified proteins. Such proteins may provoke allergies or side-effects.

In a preferred embodiment of the present invention for infection of cells and isolation of HCMV particles, different HCMV strains can be used. Strains to be used in particular can be laboratory or clinical HCMV strains.

In a further preferred embodiment the HCMV strain to be used can be Towne, AD169, TB40/E or Towne var RIT3.

A further subject-matter of the present invention relates to HCMV strains containing a functional pentameric complex. The pentameric complex is encoded in the UL128-131A gene region of the HCMV genome. They are assembled into the pentameric gH-GL-UL128-UL130-UL131A envelop complex which has been recognized as determinants for HCMV endothelial cell tropism.

In a further preferred embodiment of the present invention, the HCMV particles, preferably Dense Bodies in step (c) are isolated from the medium or from the intracellular space of the amniocyte cell.

The isolation and purification of the HCMV particles produced according to the method of the present invention is conducted via standard procedures which are known in the prior art. The kind of purification is dependent on the origin of the HCMV particles. In the case the particles have to be recovered from the inside of the cells, since the HCMV particles are still intracellularly localized, it is necessary to permeabilize the cells. This permeabilization can be conducted due to shear forces or via osmolysis. Afterwards, insoluble material such as cell membranes is separated for example via centrifugation. Centrifugation is generally used to separate cells, cell organelles and proteins. Furthermore, after the separation of other cell components it is necessary to separate distinct proteins, peptides and amino acids of different size. The purification is negatively influenced by the presence of lipids and the presence of proteases. Such deactivation of proteases is important for the purification procedure. Proteins deriving from the extracellular matrix do not have to be extracted for the purification. However, after the separation of all insoluble components, the proteins derived from the extracellular matrix are very diluted and thus only present in minor amounts compared to intracellular deriving proteins.

Preferably, the isolating in step (d) is conducted from the medium with rate velocity gradient centrifugation, density gradient differential centrifugation or zone centrifugation. Further alternative isolation methods are preferred which are suitable for the isolation of biomolecules like viruses. In particular preferred are alternative isolating steps which include the use of chromatography media that are cast as single units and result in fractionating large biomolecules like viruses.

In a preferred embodiment of the present invention, the isolating in step (d) comprises first a density gradient differential centrifugation, which is conducted from the medium to fractionate the subviral particles of the HCMV and in a second step the single subviral fractions are isolated by using a syringe and a gauge needle. Preferably, the gradients are glycerol-tartrate gradients.

In another preferred embodiment of the present invention the HCMV particles are isolated according to step d) of the method of the invention at day 2, 3, 4, 5, 6, 7, 8, 9 or 10 after infection.

In a preferred embodiment of the present invention, the permanent human amniocyte cell is grown in the lag phase, the exponential phase or the stationary phase during the time of the contacting and infecting with HCMV in step (a).

Advantageously, infection efficiency is improved if the cells are infected during the exponential—also called log—phase and the stationary phase of growth.

In a further preferred embodiment of the present invention, cells are cultivated in a growth medium optimized for infection with HCMV. Alternatively, cells are cultivated in a medium optimized for high-cell density growth of cells, and for infection with HCMV, medium is exchanged completely or is diluted with a medium optimized for infection with HCMV.

Medium optimized for infection of cells with HCMV advantageously does not contain factors preventing or reducing infection of cells with HCMV by inhibiting virus-cell binding and/or fusion. These inhibitory factors include, but are not limited to, sulfated polysaccharides, antifoaming agents, agents avoiding shear stress of cells and hydrolysates.

For infection, cell concentrations in logarithmic growth phase are at least between 3×10⁵ cells/ml and up to 1×10⁷ cells/ml. In order to prolong the log-phase beyond 1×10⁷ cells/ml additional feed supplements or process operations can be applied prior to infection to achieve a high-cell-density concentrations. These supplements or process operations include glucose, glutamine, amino acids, or avoiding metabolic waste that limit cell growth, optimizing pH and osmolarity.

The permanent human cells used in the method according to the present invention are developed via immortalization of primary human cells. Primary human cells are yielded via direct removal from the organism or a tissue derived from the organism; removed cells are cultured. Particularly preferred are primary human cells which are altered to permanent human cell lines due to the expression of cell transforming factors. Preferred primary cells are amniocytes, embryonal retinal cells as well as embryonal cells of neuronal origin.

Cell transforming factors can be T-Antigen of SV40 (Genbank Acc. No. J02400), E6 and E7 gene products of HPV (e. g. HPV16, Genbank Acc No. K02718) and E1A and E1B gene products of human adenovirus (e.g. human Adenovirus Serotyp-5, Genbank Acc. No. X02996). The primary cells become immortalized due to the expression of E1 proteins of the human adenovirus through the transfection of both nucleic acid sequences for the E1A and E1B genes. During the expression of the naturally occurring HPV, E6 and E7 can be expressed from one RNA transcript. The same applies for the expression of E1A and E1B of a naturally occurring adenovirus. The cell transforming factors, such as the adenoviral E1 gene functions exert the immortalization or transformation and thus provide the enduring ability to culture the cells.

Immortalisation of primary cells occurs by transfecting cells with nucleic acid sequences expressing the respective transforming factors. Such nucleic acid sequences can be combined on one plasmid or located on several plasmids each containing expression units for single proteins. Expression units for transforming factors each comprises a promoter, a nucleotide sequence coding for the transforming factor and a 3′ UTR. Nucleic acid molecules for transfection can also include fragments of the respective viral genome, e. g. the adenoviral genome, with the respective gene functions, e. g. E1A, E1B. The expression of the cell transforming factors can be conducted under the control of a homologous promoter or a heterologous promoter. As heterologous promotors can serve e. g. CMV (Cytomegalovirus) promotor, (Makrides, 9-26 in Makrides (ed.), Gene Transfer and Expression in Mammalian Cells, Elsevier, Amsterdam, 2003), EF-1α-promotor (Kim et al., Gene 91:217-223, 1990), CAG-Promotor (a hybrid promotor from the Immediate Early-Enhancer of human Cytomegalovirus and of a modified chicken β-actin promotor with a first intron) (Niwa et al., Gene 108:193-199, 1991), human or murine pgk- (phosphoglyceratkinase-) promotor (Adra et al., Gene 60:65-74, 1987), RSV- (Rous Sarkoma Virus-) promotor (Makrides, 9-26 in: Makrides (ed.), Gene Transfer and Expression in Mammalian Cells, Elsevier, Amsterdam, 2003) or SV40- (Simian Virus 40-) promotor (Makrides, 9-26 in: Makrides (ed.), Gene Transfer and Expression in Mammalian Cells, Elsevier, Amsterdam, 2003).

The cells become immortalized due to the transfection of the primary human cells with a nucleic acid molecule including the E1A and E1B coding nucleic acid sequences. The nucleic acid molecule including E1A and E1B nucleic acid sequences used for the immortalization of the primary cells, are preferably from human adenovirus, in particular preferred from human adenovirus serotyp-5. In a particular preferred embodiment of the present invention, the nucleic acid molecule comprises besides of the E1A and E1B coding nucleic acid sequences further nucleic acid sequences coding for the adenoviral pIX gene function. The pIX polypeptide, a viral structural protein, functions as transcription activator for several viral and cellular promotors, such as thymidine kinase and beta-globin promotor. The transcription activating function of the pIX polypeptide additionally expressed in the cell may exert an elevation of the expression rates of the recombinant polypeptide, in the case the coding sequences of the recombinant polypeptide are under the control of one of the previously mentioned promotors in the cell lines according to the present invention. An example for such a sequence is disclosed in Genbank Acc No. X02996.

In a preferred embodiment of the present invention the adenoviral gene products E1A and E1B comprise the nucleotides 1 to 4344, 505 to 3522 or nucleotide 505 to 4079 of the human adenovirus serotype-5.

In a preferred embodiment, the nucleic acid molecule for the immortalization of the primary cells comprises, in particular for amniocytes as primary cells, the adenovirus sertoype 5 nucleotide sequence of nucleotide 505 to nucleotide 4079. In a further particularly preferred embodiment of the present invention, the nucleic acid molecule used for immortalization of the primary cells, in particular of amniocytes, comprises the adenovirus serotype 5 nucleotide sequence of nucleotide 505 to nucleotide 3522. In a further particularly preferred embodiment the nucleic acid molecule used for the immortalization of primary cells, in particular of amniocytes, comprises the adenovirus serotype 5 nucleotide sequence of nucleotide 1 to nucleotide 4344, which corresponds to the adenoviral DNA in HEK-293 (Louis et al., Virology 233:423-429, 1997). Further, the immortalized human cell is able to express a viral factor which can bind to the origin of replication (ori) of a nucleic acid molecule which has been transfected into the cell. Due to this binding the replication of episomal nucleic acid molecules can be initiated. The episomal replication of nucleic acid molecules, in particular of plasmid DNA, in the cells exerts a strong augmentation of the number of copies of the transferred nucleic acid molecules and thus an elevation of the expression of a recombinant polypeptide encoded on the molecule as well as its maintenance over several cell divisions. Such a replication factor is for example the T-Antigen of Simian Virus 40 (SV40), which initiates the replication of the nucleic acid molecule, e. g. the plasmid DNA, after binding of a sequence which is designated as SV40 origin of replication (SV40 ori). The Epstein-Barr-virus protein EBNA-1 (Epstein Barr virus Nuclear Antigen-1) recognizes a so called ori-P origin of replication and catalyses the extrachromosomal replication of the ori-P including nucleic acid molecule. The T-Antigen of Simian Virus (SV40) activates not only as a replication factor the replication, but has also an activating effect on the transcription of some viral and cellular gene (Brady, John and Khoury, George, 1985, Molecular and Cellular Biology, Vol. 5, No. 6, p. 1391 to 1399).

The immortalized human cell used in the method according to the present invention is in particular an immortalized human amniocyte cell. In a preferred embodiment of the present invention, the immortalized human cell used in the method according to the present invention expresses the large T-Antigen of SV40 or the Epstein-Barr-virus (EBV) Nuclear Antigen-1 (EBNA-1). In a further preferred embodiment, the immortalized human cell used in the method according to the present invention, in particular the amniocyte cell, expresses the large T-Antigen of SV40 under the control of CAG, RSV or CMV promotor.

In a particularly preferred embodiment of the present invention, the human permanent amniocyte cells are CAP cells. In a further particularly preferred embodiment of the present invention, the human permanent amniocyte cells are CAP-T cells. These permanent human amniocyte cell lines are in particular disclosed in EP 1 230 354 and EP 1 948 789. In a further preferred embodiment, the human permanent amniocyte cells are N52.E6 cells, as disclosed in EP 1 230 354 and DE 199 55 558.

Preferably, the permanent human amniocyte cell expresses the adenoviral gene product pIX. In a particularly preferred embodiment, the permanent human amniocyte cells are CAP cells which have been transfected with a murine pgk promotor, Ad5 sequences nt. 505-3522 comprising the whole E1 region, the 3′ splice and polyadenylation signal of SV40 and the pIX region of Ad5 nt. 3485-4079. This plasmid is described in detail in EP 1 948 789.

In a preferred embodiment of the present invention, the amniocyte cell is contacted and infected in step (a) with HCMV in an amount in the range of 0.001 to 10 m.o.i., more preferably in the range of 1 to 5 m.o.i (multiplicity of infection) and most preferably of 1, 1.3, 2.5, or 5 m.o.i.

A further subject-matter of the present invention relates to the HCMV particles produced according to a method of the present invention. In a preferred embodiment of the present invention the HCMV particles obtained by the method according to the present invention comprises Dense Bodies (DB), virions and non-infectious enveloped particles (NIEPs).

In another preferred embodiment the HCMV particles obtained by the method according to the present invention comprises a fraction of NIEPs of 20 to 90%, more preferably of 50 to 90% in relation to the total protein amount of the fractions of NIEPs, virions and Dense Bodies and/or a fraction of virions of 0.5 to 50%, preferably of 1 to 20% in relation to the total protein amount of the fractions of NIEPs, virions and Dense Bodies and/or a fraction of Dense Bodies of 10 to 90%, more preferably of 40 to 80% in relation to the total protein amount of the fractions of NIEPs, virions and Dense Bodies.

Dense Bodies (DBs) are HCMV particles which are composed of pp65, gB, and gH as integral constituents. The HCMV particles represent defective viral particles which are released during infection. The HCMV particles possess an inner protein structure which is mainly composed of tegument proteins pp65 (pUL83) and pUL25. These proteins represent the most abundant proteins. The outer layer of these HCMV particles are composed of a lipid bilayer, derived from cellular membranes, in which viral glycoproteins are inserted. Further, less abundant proteins are present within the HCMV particles. Of particular importance is the protein pp65, which represent the major target protein to provoke a T lymphocyte response. Further, glycoprotein B (gB, gpUL55) and glycoprotein H (gH, gpUL75) are crucial since these glycoproteins are target structures of neutralizing antibodies.

A further subject-matter of the present invention relates to a HCMV based vaccine comprising HCMV particles according to the present invention. In a preferred embodiment the HCMV based vaccine comprises Dense Bodies.

A further subject-matter of the present invention relates to the use of HCMV particles, preferably Dense Bodies according to the present invention for the preparation of a HCMV based vaccine.

The HCMV particles produced according to the method of the present invention include HCMV proteins which are correctly folded. Thus, the protein folding is such that the appearance is comparable to the folding which occurs in a typical HCMV infection. This has the advantage that the HCMV particles included in the HCMV based vaccine are internalized by the cells of the subject as the recipient of the vaccine. After the internalization of the HCMV particles the viral proteins included in the particles are presented by antigen presenting cells (APCs). Due to the correct folding of the proteins, presentation by the APCs is conducted very efficiently. This in turn enables a strong immune response after the vaccination. The native folding of the proteins also allows the efficient induction of conformation-dependent, antiviral neutralizing antibodies, which may comprise a significant fraction of the total neutralizing-antibody capacity induced following natural HCMV infection.

Preferably, the HCMV particles, preferably Dense Bodies are placed in a pharmaceutically acceptable solution for the preparation of a HCMV based vaccine.

The HCMV based vaccine according to the present invention can be provided with one or more additional substances, such as stabilizers, neutralizers, carrier or substances for preservation. Such substances are for example formaldehyde, thiomersal, aluminium phosphate, acetone and phenol. Furthermore, the HCMV based vaccine according to the present invention may also include adjuvants to improve the immune stimulatory effect of the vaccine. Preferably, such adjuvants do not exert itself a pharmacological effect. These adjuvants may serve as solubilizer, emulsion or mixtures thereof. Adjuvants are for example mineral salts, squalen mixtures, muramyl peptides, saponin derivatives, preparations of Mycobacteria cell wall, distinct emulsions, monophosphoryl-lipid-A, mycolic acid derivatives, non-ionic block-copolymer tensides, quil A, subunit of cholera toxin B, polyphosphazene and its derivatives, immune stimulatory complexes, cytokine adjuvants, MF59 adjuvants, lipid adjuvants, mucosal adjuvants, distinct bacterial exotoxines, and distinct oligonucleotides and PLG.

A further subject-matter of the present invention relates to the HCMV particles according to the present invention, preferably Dense Bodies for use in the preparation of a therapeutic or diagnostic agent for the prevention or treatment of HCMV related disease.

### LIST OF REFERENCES

1. Andreoni, M., M. Faircloth, L. Vugler, and W. J. Britt. 1989. A rapid microneutralization assay for the measurement of neutralizing antibody reactive with human cytomegalovirus. J. Virol. Methods 23:157-167.
2. Craighead, J. E., R. E. Kanich, and J. D. Almeida. 1972. Nonviral microbodies with viral antigenicity produced in cytomegalovirus-infected cells. J. Virol. 10:766-775.
3. Digel, M., K. L. Sampaio, G. Jahn, and C. Sinzger. 2006. Evidence for direct transfer of cytoplasmic material from infected to uninfected cells during cell-associated spread of human cytomegalovirus. J. Clin. Virol. 37:10-20. doi:S1386-6532(06)00160-0 [pii]; 10.1016/j.jcv.2006.05.007 [doi].
4. Hoehn, H., E. M. Bryant, L. E. Karp, and G. M. Martin. 1974. Cultivated cells from diagnostic amniocentesis in second trimester pregnancies. I. Clonal morphology and growth potential. Pediatr. Res. 8:746-754. doi:10.1203/00006450-197408000-00003 [doi].
5. Pepperl, S., J. Münster, M. Mach, J. R. Harris, and B. Plachter. 2000. Dense bodies of human cytomegalovirus induce both humoral and cellular immune responses in the absence of viral gene expression. J. Virol. 74:6132-6146.
6. Pepperl-Klindworth, S. and B. Plachter. 2006. Current perspectives in vaccine development, In: M. J. Reddehase (ed.), Cytomegaloviruses: Molecular Biology and Immunology. Caister Academic Press Ltd, Wymondham, Norfolk, U.K.
7. Scrivano, L., C. Sinzger, H. Nitschko, U. H. Koszinowski, and B. Adler. 2011. HCMV spread and cell tropism are determined by distinct virus populations. PLoS. Pathog. 7:e1001256. doi:10.1371/journal.ppat.1001256 [doi].
8. Stinski, M. F. 1976. Human cytomegalovirus: glycoproteins associated with virions and dense bodies. J. Virol. 19:594-609.
9. Topilko, A. and S. Michelson. 1994. Hyperimmediate entry of human cytomegalovirus virions and dense bodies into human fibroblasts. Res. Virol. 145:75-82.
10. Varnum, S. M., D. N. Streblow, M. E. Monroe, P. Smith, K. J. Auberry, L. Pasa-Tolic, D. Wang, D. G. Camp, K. Rodland, S. Wiley, W. Britt, T. Shenk, R. D. Smith, and J. A. Nelson. 2004. Identification of proteins in human cytomegalovirus (HCMV) particles: the HCMV proteome. J. Virol. 78:10960-10966.
11. Wills, M. R., A. J. Carmichael, K. Mynard, X. Jin, M. P. Weekes, B. Plachter, and J. G. Sissons. 1996. The human cytotoxic T-lymphocyte (CTL) response to cytomegalovirus is dominated by structural protein pp65: frequency, specificity, and T-cell receptor usage of pp65-specific CTL. J. Virol. 70:7569-7579.

The following examples describe the present invention, however, these examples should not be considered as limiting.

### EXAMPLE 1

### Infection of Adherent CAP Cells and Human Foreskin Fibroblasts (HFF) with BAC-Derived Virus RV-TB40/E BAC7

For this example adherent CAP cultivated in Opti-Pro medium (Life Technologies/Gibco) without serum and HFF (control) cells were seeded in 10 cm culture dishes (78 cm², 5×10⁵ cells). Coverslips were inserted into these dishes. Following overnight incubation, cells were infected with an m.o.i. of 1 with TB40/E BAC7 in a total volume of 4 mL. After an adsorption period of 1.5 hours, cells were replenished with medium to a total volume of 10 mL for each dish. A mock-infected control was carried along for each cell type. One day post infection, cover slips were collected, washed with 1×PBS and fixed in acetone p.a. (AppliChem; A1600,2500) for 1 hour at room temperature. Subsequently, the cell membrane was permeabilized by several rounds of rinsing in 1×PBS/0.1% Triton (3 times; TritonX 100; Roth). After that, cells were incubated with murine monoclonal antibody p63-27, directed against the HCMV protein IE1-pp72 [(ppUL123; (1)] for 1 h, 37° C. Cells were then washed several times in 1×PBS/0.1% Triton (3 times) and incubated with a FITC-labeled secondary antibody, directed against mouse immunoglobulin for 1 hour, 37° C. For staining of the nuclei, DAPI (4′,6-Diamidin-2-phenylindol; Invitrogen) was diluted 1:5,000 in 1×PBS and was added to the cover slips in a volume of 150 μL (together with the secondary antibody, final volume of 3004). Cells were then kept at RT in the dark for 10 minutes. After that, cover slips were rinsed three times in 1×PBS/0.1% TritonX 100 and once with distilled water. After that, coverslips were transferred to glass slides and fixed with Mowiol (Sigma Aldrich; 81381-250 g; 20 g dissolved in 80 mL 1×PBS and 40 mL glycerol). Glass slides were kept in the dark at room temperature to allow drying until inspection. FIG. 3A and FIG. 3C, indirect immunofluorescence of infected CAP (Opti-Pro) cells stained for IE1-pp72 expression (different magnifications). FIG. 3B and FIG. 3D, indirect immunofluorescence of infected HFF, stained for IE1-pp72 expression (different magnifications).

### EXAMPLE 2

### Infection of Adherent CAP Cells with Virus RV-TB40/E-delUL16EGFP

For this example adherent HFF (control) and CAP cells (Opti-Pro with serum or VP-SFM [Life Technologies/Gibco)] without serum) were seeded (5×10⁵ cells) in 75 cm² culture bottles. Following overnight incubation, cells were infected with an m.o.i. of 1 with virus RV-TB40/E-delUL16EGFP (FIG. 1) in a total volume of 4 mL. After an adsorption period of 1.5 hours, cells were replenished with medium to a total volume of 10 mL for each flask. Three days post infection, infected cells were inspected in vivo, using a Leitz DMIRB microscope (luminous source: Leica 220V; 50/60 HZ, HG 50 W). FIG. 4A shows a direct fluorescence of infected adherent CAP (Opti-Pro), cultivated in the presence of serum. FIG. 4B shows a direct fluorescence of infected adherent CAP (VP-SFM), cultivated in the absence of serum.

The results shown in FIGS. 3 and 4 provided the proof that HCMV can penetrate CAP cells and initiate its immediate—early gene expression. The latter is considered essential for lytic replication. About 20-40% of adherent CAP cells proved to be infectable (CAP Opti-Pro).

### EXAMPLE 3

### Infectability of Adherent CAP Cells by HCMV at 24 Hours of Infection Analyzed by Luciferase Expression

Adherent CAP (cultivated in Opti-Pro without serum) cells and HFF (control) were seeded in 96-well plates (1.5×10⁴ cells per well in 25 μL). After overnight incubation, cells were infected with RV-TB40/E-BAC4 deltaUL5-9luc (FIG. 2A, luciferase expression under the control of the SV40 promoter) and RV-TB40/E-UL84luc (FIG. 2B, luciferase expression under the control of the early UL84 HCMV promoter), respectively at different dilutions (as indicated). After an incubation period of 24 hours, cells were cooled to room temperature. 125 μL of BrightGlow™ reagent (Promega Bright Glow™ luciferase assay system) were added and cells were incubated for 2 minutes. Luciferase activity was measured in a Berthold Detection System Orin II Microplate Luminometer. FIG. 5A is a representation of luciferase activity, measured following a 24 hour infection with RV-TB40/E deltaUL5-9luc. FIG. 5B is a representation of luciferase activity measured following a 24 hour infection with RV-TB40/E-UL84luc.

### EXAMPLE 4

### Infectability of Adherent CAP (Opti-Pro with Serum) Cells and CAP (VP-SFM without Serum) by HCMV at 48 Hours of Infection Analyzed by Luciferase Expression

The experimental setup was as described in example 3. FIG. 6A is a representation of luciferase activity, measured following a 48 hour infection with RV-TB40/E deltaUL5-9luc (luciferase expression under control of the SV40 promoter). FIG. 6B is a representation of luciferase activity, measured following a 48 hour infection with RV-TB40/E-UL84luc (luciferase expression under the control of the early UL84 HCMV promoter).

The results shown in FIGS. 5 and 6 confirmed the findings of FIGS. 3 and 4 with respect to the infectability of CAP cells by HCMV. The result of FIGS. 5B and 6B, in particular, showed that early promotors of HCMV (UL84) are active in adherent CAP cells cultivated in the presence (Opti-Pro) or absence (VP-SFM) of serum.

### EXAMPLE 5

### Determination of Viral Genome Copies after Infection of Adherent CAP (Opti-Pro, VP-SFM) and HFF

Virus used for infection had to be normalized to genome copies in infected cells at 6 hours post infection. For this, 0.5×10⁶ adherent CAP cells cultivated in the presence (Opti-Pro) or absence (VP-SFM) of serum and 0.5×10⁶ HFF were seeded in 10 cm dishes. After overnight incubation, cells were infected in 3 mL culture medium, using different virus dilutions (5 μL, 10 μL, 50 μL, 100 μL, 500 μL) of a culture supernatant from TB40/E BAC7. After an adsorption period of 1.5 hours, dishes were replenished with additional 7 mL of culture medium. At 6 hours after infection, supernatant was discarded. Cells were washed two times with 3 mL of 1×PBS. Following that, 2.5 mL of trypsin were added to HFF and incubated for 5 minutes at 37° C. For CAP cultures, 3 mL of trypsin was applied, spread and discarded directly afterwards without incubation. After that, 2.5 to 3 mL of medium containing serum was added to stop the reaction. Following that, cells that were detached from the support, were centrifuged at 472×g for HFF and at 134×g for CAP cells for 5 minutes. The cell pellet was resuspended in 500 μL 1×PBS. Cells were then counted and adjusted to 1×10⁶/mL. For this, cells were again centrifuged at 472×g (HFF) or at 134×g (CAP) and were then resuspended in the appropriate volume of 1×PBS. DNA was isolated using the “High Pure Viral Nucleic Acid Kit, Roche©” according to the manufacturer's instructions. Viral DNA concentration was determined in each sample using the ABI Prism 7700 Sequence Detection System (Serial-No.: 100000740).

### Reagents:

### Hot Star Taq Polymerase (Quiagen; 5 U/μL)

### Mastermix

10×PCR Buffer, including 15 mM MgCl₂, 25 mM MgCl₂, 2 mM dNTPs, 0.3 μM CMV-forward primer and reverse primer (Eurofins MWG Operon), 1 μM probe (TIB MOLBIOL), 100 μM ROX (6-Carboxy-X-rhodamin) in LiChrosolv water for chromatography (Merck, Cat.-No. 1.15333.1000).

1.2 mL mastermix and 12.5 μL polymerase were mixed.

Reactions were performed in 96 well plates in triplicates. The first vial received 135 μL master mix. 15 μL template was subsequently added. 50 μL each of that mixture were transferred to the second and third vial. The tubes were sealed and applied to the ABI Prism 7700 Sequence Detection System. Program details were as follows:

| | |
|---|---|
| 50° C. for 2 min | |
| 95° C. for 5 min | (activation of Hot Star Taq) |
| 45 cycles of | |
| 94° C. for 15 sec | (denaturation) |
| 60° C. for 1 min | (annealing, elongation) |
| 4° C. | End of reaction |

Results were calculated in genomes/mL. Using these results, volumes of virus stocks were determined which were necessary to provide 4 genome/copies per cell at 6 hours of infection.

To determine replication kinetics in infected cells, 0.5×10⁶ HFF and CAP cells, respectively were seeded in 9×10 cm dishes. After incubation overnight at 37° C., cells were infected with TB40/E BAC7 to result in 4 genome copies of viral DNA per cell. After different intervals, cells were collected and counted. DNA was isolated and viral genome copies/cell were determined using the methodology as described above. FIG. 7 shows the total number of viral genome copies in the dish in relation to time of infection.

The results—shown in FIG. 7—proved that HCMV can replicate its genome in CAP cells, irrespective to whether or not serum is added to the medium.

### EXAMPLE 6

### Odyssey® Immunoblot Analysis of the Expression of Viral Proteins in Adherent CAP Cells

1.8×10⁶ adherent CAP cells (Opti-Pro with serum) were seeded in 175 cm² cell culture flasks. For control, 1.5×10⁶ MRC5 were seeded in parallel. After overnight incubation, cells were infected with RV-40E-deltaUL16EGFP at a multiplicity of infection of 10 (CAP) or 1 (MRC5), respectively. At six days after infection, culture medium was removed and cells were washed once with 1×PBS. 5 mL of trypsin was added and immediately removed (CAP) or left on the cells at 37° C. for 5 minutes (MRC5). Addition of medium with serum was used to stop the reaction. Cells were centrifuged at 472×g (MRC5) or 134×g (CAP), 1×PBS was repeat washing of cells. After that, cells where counted. After another centrifugation step, the cells were resuspended in Laemmli-Buffer (125 mM Tris-Base, 2% vol/vol β-mercaptoethanol, vol/vol 10% glycerin, 1 mM EDTA pH 8.0, 0.005% vol/vol bromphenol-blue, H₂O_{dest.}) to result in 2×10⁵cells/10 μL. Samples were boiled for 10 minutes, centrifuged at 1,300 g for three minutes, partitioned and stored at −20° C. until further use.

For the preparation of the sample “CAP (Opti-Pro 14 dp.i. SN 40 μL)”, two 175 cm² flasks, containing an initial seed of 1.8×10⁶ adherent CAP cells each were infected with RV-TB40/E-delUL16EGFP at an multiplicity of infection of 10 and were cultivated for 14 days. A culture medium exchange was performed one day after infection. 14 days after infection, culture supernatant was collected and centrifuged at 1647×g for 10 minutes to remove cell debris. The supernatants were transferred to ultracentrifuge tubes and were centrifuged at 131.250×g, 10° C. for 70 minutes in a Beckman Coulter Optima L-90K ultracentrifuge (Serial-No.: COL08L10). The supernatant was then removed carefully. The pellet was thoroughly resuspended in 40 μL of Laemmli-Puffer. Again the sample was boiled for 10 minutes and was then centrifuged at 1,300×g for 3 minutes. Samples were stored at −20° C. until further use.

10% SDS-polyacrylamide gels were used for the separation of the proteins in the samples. Separation gels: 10 ml distilled water, 6.25 ml Tris 1.5 M (pH 8.8), 8.3 mL Gel 30 (Rotiphrese® Gel 30, Roth) 250 μL 10% SDS, 250 μL 10% APS, 15 μL TEMED.

Stacking gels: 4.30 mL distilled water, 0.75 mL Tris 1M (pH 6.8), 1.05 mL Gel 30 (Acrylamide), 0.624 μL 10% SDS, 0.62 μL 10% APS, 7.5 μL TEMED.

After polymerisation, gels were mounted on a vertical gel chamber (Hoefer SE 600 Series Electrophoresis Unit; U.S. Pat. No. 4,224,134). The running buffer (upper and lower buffer chamber) contained 1×PAGE buffer (200 mL 5×PAGE-Puffer [25 mM Tris-Base, 192 mM glycine, 0.1% SDS]+800 mL distilled water). 10 μL pre-stained protein marker (PeqGOLD Protein Marker IV from Peqlab; cat. no. 27-2110) was applied to the first slot; the samples were applied to the other slots. Proteins were separated overnight at 4.35V/cm (50 V, 400 mA and 100 W; Electrophoresis Power Supply—EPS 600; Pharmacia Biotech).

The following day, the glass plates were removed and the gels were rinsed in water. The PVDF filter (PVDF-Membrane: “Millipore” Immobilion, Transfer Membrane, Immobilion-FL, cat. no. IPFL00010) was cut in appropriate pieces and was rinsed in methanol for 5 minutes, briefly rinsed in water and then equilibrated for 20 minutes in transfer buffer/10% vol/vol methanol (25 mM Tris, 192 mM glycine, 10% methanol, 2 L distilled water). The gel was also incubated in transfer buffer for 20 minutes. The initial layer on the semi-dry blot apparatus (Hölzel) consisted of three layers of chromatography paper (Chromatography Paper Whatman®) which was pre-soaked in transfer buffer. Next, the equilibrated PVDF-membrane was applied, avoiding the generation of air-bubbles. After that the gel was applied, again avoiding air-bubbles. The final stack consisted of 3 layers of chromatography paper. The transfer was performed using 600V, 400 mA for 75 minutes. After that, the apparatus was dismantled and the PVDF-membrane with the transferred proteins was air-dried for 1-2 hours in a fume hood. The membrane was then briefly rinsed in methanol, followed by rinsing it in H₂O. After an incubation in 1×PBS for a few minutes, blocking reagent (5% w/v dry milk powder [Roth, Art. Nr. T145.2] in 1×PBS) was applied and the filters were incubated on a tumbling device for 1 hour.

The primary murine monoclonal antibody 65-33, directed against pp65 (ppUL83) of HCMV (obtained from Prof. W. Britt, UAB, Birmingham, Ala., USA) was diluted 1:500 in blocking reagent/0.1% v/v Tween100. Primary goat polyclonal antibodies against pp71 (ppUL82; vC-20, Santa Cruz Biotechnology, Heidelberg) were diluted 1:200 in blocking reagent/0.1% v/v Tween100. Membranes were incubated with the primary antibody in film wraps over night at room temperature, avoiding trapping for air bubbles. The next day, the antibody was removed and the filters were washed three times for 10 minutes in 1×PBS/0.2% v/v Tween100. After that, secondary antibodies were applied in a dilution of 1:5.000 in blocking reagent/0.1% v/v Tween100/0.01% v/v SDS. Secondary antibodies were IR800 donkey-anti-goat or IR800 goat-anti-mouse (Rockland). Incubation was performed in the dark for two hours at room temperature. After that, the secondary antibody was removed by 2 washing steps for 10 minutes in 1×PBS/0.2% Tween100 and 1 washing step for 10 minutes in 1×PBS in the dark. Evaluation of the results was performed with an “Odyssey Imager” (LI-COR; Lincoln, Nebr., USA).

The results of the immunoblots, illustrated in FIG. 8, showed that pp65 and pp71 are detectable in infected CAP cells (cultivated in Opti-Pro with serum) at 6 days post infection and that pp71 was also faintly detectable in the supernatant of infected CAP cells, collected at 14 days post infection.

### EXAMPLE 7

### Release of Viral Genomes into the Cell Culture Supernatant of Infected CAP Cells

2×1.8×10⁶ HFF or 2×1.8×10⁶ CAP cells (cultivated in Opti-Pro with serum) or 2×1.6×10⁶ CAP cells (cultivated in VP-SFM without serum) were seeded in 175 cm² culture flasks in 20 mL of the respective culture medium. Cells were infected with RV-TB40/E-delUL16EGFP the following day in a total volume of 5 mL. For HFF and CAP (VP-SFM), multiplicity of infection of 1 for CAP (Opti-Pro) or 0.1 for HFF and CAP (VP-SFM) was used. After an adsorption period of 1.5 hours, 15 mL of additional medium was added to the flasks. After that, 1.5 mL culture supernatant was removed from each flask and stored at −20° C. The DNA concentration measured in these samples was taken as base-line value (see below). At day 1 of infection, the culture medium in all flasks was replaced by fresh medium. At days 2, 3, 5, and 6 (or days 2, 3, and 6 for CAP VP-SFM), 1.5 mL of culture supernatant was sampled from each flask and replaced by 1.5 mL of fresh medium. At day 6, all cells were passaged and 2×1.8×10⁶ of both HFF and CAP (Opti-Pro or VP-SFM) cells were seeded in new 175 cm² flasks. Surplus cells were discarded. Again, 1.5 mL of culture medium was sampled at days 8, 10, 12, 13, and 14 (or at days 8, 10, 13, and 14 for CAP VP-SFM).

The DNA contained in 200 μL of each sampled specimens was extracted using the “High Pure Viral Nucleic Acid Kit, ROCHE©” Kit according to the manufacturer's instructions. Quantification of viral genomic DNA was performed using ABI Prism 7700 Sequence Detection System, Applied Biosystems, (Serial-No.: 100000740).

### Reagents:

Hot Star Taq polymerase (Quiagen; 5 U/μL)

### Mastermix

10×PCR Buffer, including 15 mM MgCl₂, 25 mM MgCl₂, 2 mM dNTPs, 3 μM CMV-forward primer and reverse primer (Eurofins MWG Operon), 1 μM probe (TIB MOLBIOL), 100 μM ROX (6-Carboxy-X-rhodamin) in LiChrosolv water for chromatography (Merck, Cat.-No. 1.15333.1000).

1.2 mL Mastermix and 12.5 μL polymerase were mixed.

Reactions were performed in 96 well plates in triplicates. The first vial received 135 μL master mix. 15 μl template was subsequently added. 50 μL each of that mixture was transferred to the second and third vial. The tubes were sealed and applied to the ABI Prism 7700 Sequence Detection System. Program details were as follows:

| | |
|---|---|
| 50° C. for 2 min | |
| 95° C. for 5 min | (activation of Hot Star Taq) |
| 45 cycles of | |
| 94° C. for 15 sec | (denaturation) |
| 60° C. for 1 min | (annealing, elongation) |
| 4° C. | End of reaction |

The results were calculated as genome copies/20 mL of culture supernatant and are shown in FIG. 9. This provided proof that viral genomes were released over a prolonged period from infected CAP (Opti-Pro) cells.

### EXAMPLE 8

### Release of Viral Genomes into the Cell Culture Supernatant of Infected CAP Cells (with Medium Exchange 1 Day after Infection)

1.5×10⁶ MRC5 or 1.8×10⁶ CAP (Opti-Pro) cells were seeded in 175 cm² culture flasks in quadruplicates in 20 mL of the appropriate culture medium. Cells were infected with RV-TB40/E-delUL16EGFP the following day in a total volume of 5 mL. Infection was with an m.o.i. of 5 or 10, respectively, for CAP (Opti-Pro), and 1 for MRC5 cells. After an adsorption period of 1.5 hours, 15 mL of additional medium was added to the flasks. After that, 1.5 mL culture supernatant was removed from each flask and stored at −20° C. The DNA concentration measured in these samples was taken as basal value (see below). One day after infection, the culture medium in all flasks was removed, cells were rinsed in 1×PBS and medium was replaced by fresh medium. At days 4, 5, 6, 8, 11, 12, and 14, 1.5 mL of culture supernatant was sampled from each flask and replaced by 1.5 mL of fresh medium. Samples were stored at −20° C. until further use.

The DNA contained in 200 μL of each sampled specimens was extracted using the “High Pure Viral Nucleic Acid Kit, ROCHE©” Kit according to the manufacturer's instructions. Quantification of viral genomic DNA was performed using ABI Prism 7700 Sequence Detection System, Applied Biosystems (Serial-No.: 100000740).

### Reagents:

Hot Star Taq polymerase (Quiagen; 5 U/μL)

### Mastermix

10×PCR Buffer, including 15 mM MgCl₂, 25 mM MgCl₂, 2 mM dNTPs, 3 μM CMV-forward primer and reverse primer (MWG), 1 μM probe (TIB MOLBIOL), 100 μM ROX (6-Carboxy-X-rhodamin) in LiChrosolv water for chromatography (Merck, Cat.-No. 1.15333.1000).

1.2 mL Mastermix and 12.5 μL polymerase were mixed.

Reactions were performed in 96 well plates in triplicates. The first vial received 135 μL Mastermix. 15 μl template was subsequently added. 50 μL each of that mixture was transferred to the second and third vial. The tubes were sealed and applied to the ABI Prism 7700 Sequence Detection System. Program details were as follows:

| | |
|---|---|
| 50° C. for 2 min | |
| 95° C. for 5 min | (activation of Hot Star Taq) |
| 45 cycles of | |
| 94° C. for 15 sec | (denaturation) |
| 60° C. for 1 min | (annealing, elongation) |
| 4° C. | End of reaction |

The results were calculated as genome copies/20 mL of culture supernatant and are shown in FIG. 10.

The results provided proof that viral genomes were released over a prolonged period from infected adherent CAP cells cultivated in Opti-Pro in the presence of serum.

### EXAMPLE 9

### Proof of Release of Infectious Virus from CAP Cells

0.5×10⁶ HFF, or 1.2×10⁶ adherent CAP cells cultivated in Opti-Pro with serum, or 1×10⁶ adherent CAP cells cultivated in VP-SFM without serum, respectively, were seeded in 75 cm² culture flasks and incubated overnight. Cells were infected with RV-TB40/E-delUL16EGFP the following day at m.o.i.s of 0.5, 1, and 5.1-1.5 mL culture supernatant was collected at days 3, 6, and 7 and stored at −80° C. until further analysis. 3×10⁵ HFF were seeded in 25 cm² culture flasks and incubated overnight. The following day, HFF were infected with the supernatants. FIG. 11 shows an example of the direct GFP fluorescence, detectable upon microscopic inspection.

This experiment proved that infectious virus was released from infected CAP cells.

### EXAMPLE 10

### Release of Infectious HCMV into the Culture Supernatant of Infected CAP Cells, Measured by Plaque Assay

2×1.5×10⁶ MRC5 cells or 4×1.8×10⁶ adherent CAP cells (cultivated in Opti-Pro with serum) were seeded in 175 cm² culture flasks in 20 mL of the appropriate culture medium. Cells were infected with RV-TB40/E-delUL16EGFP the following day in a total volume of 5 mL. For MRC5, an infectious dose of 1 (m.o.i.), for CAP (Opti-Pro) an infectious dose of 5 or 10 (m.o.i.) was used. After an adsorption period of 1.5 hours, 15 mL of additional medium was added to the flasks. At day 1 of infection, the culture medium in all flasks was replaced by fresh medium. 1.5 mL of medium was collected as initial sample and was replaced by 1.5 mL of fresh medium. At days 6, 11, and 14, 1.5 mL of culture supernatant was sampled from each flask and replaced by 1.5 mL of fresh medium.

For measurement of infectious virus in the samples, a standard plaque assay was performed on HFF. For this, 3×10⁵ HFF were seeded in 2 mL of the culture medium in each well of 6-well plates (5 plates). Cells were cultured overnight as described above. 1 mL of the samples was applied to the cells the following day. Supernatant from infected MRC5 cells was diluted by 1:10, supernatant from infected CAP cells was used undiluted. Adsorption was for 1.5 hours at 37° C. The supernatant was then discarded and 4 mL of overlay medium was applied to the cells to prevent viral spread. The plates were kept at room temperature for 30 minutes to allow solidification of the overlay. Following that, plates were incubated for 6 days at 37° C. as described above. After that, the overlay was carefully removed and crystal violet solution was applied (crystal violet from Roth [25 g] 1% cristal violet/50% Ethanol/H₂O_{dest}).

After an incubation of 10 minutes, the liquid was removed and wells were rinsed with tap water until the staining appeared appropriate. Plates were dried by leaving them on the shelf for one day. Plaques were then counted using a Leitz DMIRB microscope. The results of the plaque assay are shown in FIG. 12.

Preparation of overlay medium.

Prepare agarose stock:

Ad 1.5 g agarose (Gibco) to 50 mL of 1×PBS.

Heat this in a microwave oven.

Autoclave

Apportion 5 mL each into 50 mL Falcon tubes under sterile conditions.

Store at 4° C. until use.

To prepare overlay with 0.3% w/v agarose, 5 mL of stock agarose was briefly heated in a microwave oven and cooled to 42° C. in a water bath. In parallel, culture medium (MEM) was also heated to 42° C. The solutions were allowed to cool at room temperature for 15 minutes (final temperature of 39-40° C.). After adsorption, 5 mL agarose and 45 mL of the medium were mixed and were applied to the cells to prevent spread.

The results as depicted in FIG. 12 showed that CAP (Opti-Pro) cells released infectious virus for a prolonged period of time.

### EXAMPLE 11

### Analysis of the Induction of Apoptosis by HCMV Infection of CAP-Cells

3×10⁴ CAP (Opti-Pro) cells were seeded in a 1μ-Slide 8 well Coated (poly-L-lysine) Microscopy Chamber (Lot: 120502/3; Ibidi, Martinsried, Germany) in each chamber in a volume of 500 μL medium each. Incubation was over night at 37° C./5% CO₂ in a humidified atmosphere. 4 of 8 chambers were infected with RV-TB40/E-delUL16EGFP at an m.o.i. of 1 in a volume of 100 μL. 4 of 8 chambers were carried along as mock controls. Fixation was performed at 3 days post infection. For this, culture supernatant was discarded and the cells were washed once in cold 1×PBS. Following that, paraformaldehyde [(P6148-500 g; #109K1434; Sigma-Aldrich)+100 mL 1×PBS] was added and cells were incubated for 20 minutes. After that, paraformaldehyde was removed and the cells were washed 3 times in 1×PBS. Cells were then resuspended in 1×PBS and stored at 4° C. until analysis. Cells were then stained with the “In Situ Cell Death Detection Kit, Fluorescein (Roche, Cat. No. 1 684 795) according to the manufacturer's instructions. Analysis was performed by inspection through an Axiophot microscope (Zeiss), using fluorescent light. No indication of apoptosis following infection could be detected; infected and mock-infected cells showed equal fluorescence signals.

### EXAMPLE 12

### Titration of Virus Stocks

1.8×10⁶ HFF were seeded in a 175 cm² culture flask and were incubated over night at 37° C./5% CO₂ in a humidified atmosphere. The following day, the cells were infected with 1 mL virus stock. Virus supernatant was collected at day 7 after infection and used to infect 10-20 175 cm² culture flasks with 1.8×10⁶ HFF. 7 days after that, culture supernatant was again collected and apportioned in volumes of 1-1.5 mL. Virus stocks were frozen at −80° C. until further use.

For titration, 5×10³ in 50 μL HFF were seeded in each well of a 96 well-plate and incubated overnight. 10 fold serial dilutions of the respective virus stocks were applied to the different wells. Each sample was tested in quadruplicate. Incubation was for 2 days at 37° C./5% CO₂ in a humidified atmosphere. After that, supernatant was discarded and cells were washed with 1×PBS. Fixation was with 100 μL 96% vol/vol ethanol. After that, ethanol was discarded and cells were washed again with 1×PBS.

Residual PBS was removed by tapping the plate on a towel. Subsequently, 50 μL undiluted supernatant of the antibody p63-27 (IE1-pp72) was applied to each well. Incubation was for 1 hour at 37° C. in a wet chamber. After that the primary antibody was removed by tapping the plate on a towel and the plate was rinsed again with 1×PBS. After that, an anti-mouse antibody, coupled to horse-radish peroxidase, was applied. Incubation was again for 1 hour at 37° C. in a wet chamber. After the removal of the secondary antibody solution and two PBS-washing steps, AEC substrate was applied. AEC substrate was diluted 1:20 in acetate-buffer and was subsequently filtrated through a MN 615 Ø185 mm filter paper (Macherey-Nagel). H₂O₂ (hydrogen peroxide 30% Art.-Nr. 8070.1; Roth) was added to the substrate at a dilution of 1:1,000 immediately before application. Incubation was for 1 hour at 37° C. in a wet chamber. After removal of the substrate, cells were again rinsed twice with PBS and were stored at 4° C. until inspection. The number of positive nuclei was then taken as a measure for infectivity (m.o.i.).

Solutions:

Acetate-buffer: 13.6 g Na-Acetat×3 H₂O
2.88 mL glacial acetic acid
ad 1000 mL H₂O
pH=4.9

AEC-Stock:
Apply 10 AEC-tablets 200 mg) in a 50 ml Falcon tube.
(3-Amino-9-Ethyl-Carbazole (AEC) Tablets (No. 205-057-7, Sigma, Cat.-No. A6926)
dissolve in 50 ml DMF (n,n-Dimethylformamide; Sigma, Cat.-No. D4254, 250 ml) by vortexing
dispense in 1 mL Aliquots in 1.5 mL-tubes; store at: −20° C.

### EXAMPLE 13

### Evaluation of DB Production with Adherent CAP Cells

To evaluate the capacity of adherent CAP cells for the production of Dense Bodies (DB), cells were seeded at a density of 3.6×10⁶ cells in 20 mL Opti-Pro medium with serum in 175 cm² tissue culture flasks.

Two days after seeding, cells were infected with a derivative of the Towne strain of human cytomegalovirus (HCMV), repaired for the expression of a functional UL130 protein (Towneᵣₑₚ). For infection, the medium from 10 flasks was discarded and cells were infected with 4 ml medium containing Towneᵣₑₚ in a concentration to result in an m.o.i. of 2.5 and further incubated for 1.5 hours in the incubator. Following that, 16 mL of medium was supplemented to each flask.

After 7 days, the culture supernatants were collected. Supernatants of ten flasks were combined and centrifuged (1300×g, 10 min., room temperature) or cells were first scraped with a cell scraper and the combined cell suspensions were centrifuged.

After that, the supernatant was collected and centrifuged at 100,000×g (70 min., 10° C.) in a SW32Ti rotor in a Beckman Optima L-90K ultracentrifuge. Meanwhile, the gradients were prepared by mixing 4 mL 35% sodium-tartrate solution with 5 mL 15% sodium-tartrate/30% Glycerin-solution in 0.04 M Sodium-phosphate buffer pH7.4, using a gradient mixer and Beckman Ultra-Clear™ centrifuge tubes (14×89 mm). Following centrifugation, the pellets were resuspended in 1000 μl 1×PBS and applied on top of one gradient. Centrifugation was performed at 91,000×g (60 min., 10° C.) in a SW41 rotor.

After centrifugation, the bands, corresponding to NIEPs (non-infectious enveloped particles), virions and DB (Dense Bodies) were visualized by light scattering and collected from the gradient, using a syringe and a 80 G×1.5″-gauge needle.

Each sample was supplemented with 1×PBS to give a total volume of 10 mL. Centrifugation was then performed at 99,000×g (90 min., 10° C.) in a SW41 rotor.

Following that centrifugation, the pellets were resuspended in 50 μl (virions, DB) or 100 μl (NIEPs) 1×PBS. 15 μl were taken for further measure of protein content and stored at −80° C. until further use. The residual samples were stored in aliquots.

The protein concentrations in the samples were evaluated by using the Pierce® BCA Protein Assay Kit (Thermo Scientific, Order-No.: 23225) according to the manufacturer's instructions.

10% SDS-polyacrylamide gel was used for the separation of the proteins in the samples. Separation gels: 10 mL distilled water, 6.25 mL Tris 1.5 M (pH 8.8), 8.3 mL Gel 30 (Rotiphorese® Gel 30, Roth) 250 μL 10% SDS, 250 μL 10% APS, 15 μL TEMED. Stacking gels: 4.30 mL distilled water, 0.75 mL Tris 1M (pH 6.8), 1.05 mL Gel 30 (Rotiphorese® Gel 30, Roth), 0.62 μL 10% SDS, 0.62 μL 10% APS, 7.5 μL TEMED. After polymerization, gels were mounted on a vertical gel chamber (Hoefer SE 600 Series Electrophoresis Unit; U.S. Pat. No. 4,224,134). The running buffer (upper and lower buffer chamber) contained 1×PAGE buffer (200 mL 5×PAGE-Puffer [25 mM Tris-Base, 192 mM glycine, 0.1% SDS]+800 mL distilled water). 2 μl pre-stained protein marker (PeqGOLD Protein Marker IV from Peqlab; cat. no. 27-2110) were applied. Proteins were separated over night at 4.35 V/cm (50 V, 400 mA and 100 W; Electrophoresis Power Supply—EPS 600; Pharmacia Biotech). The glass plates were removed the following day and the gels were rinsed in water.

Silver staining of the proteins was performed using the Roti®-Black P-silver staining kit for proteins (Roth, order-No. L533.1) according to the manufacturer's instructions.

The results of this experiment are shown in FIG. 13. The results provided proof that HCMV-infected adherent CAP cells release DB, virions and non-infectious enveloped particles (NIEPs).

### EXAMPLE 14

### Evaluation of DB Production with Suspension CAP Cells

For cultivation of suspension CAP cells and infection with HCMV a medium suitable for HCMV infection has to be used, e.g. FreeStyle (Life Technologies/Gibco) or CAP-T Express (CEVEC Pharmaceuticals GmbH). To evaluate the capacity of suspension CAP cells for the production of Dense Bodies (DB), cells were seeded at a density of 2.5×10⁵ cells/mL in CAP-T Express (CEVEC Pharmaceuticals GmbH) serum-free suspension medium in 125 mL Erlenmeyer flasks in a total volume of 20 mL per flask. Cells were shaken in a Corning LSE orbital shaker at 260 rpm in an incubator (CO₂ 5%; humidity 80%) at 37° C. for 24 hours. The following day, cells were infected with a derivative of the Towne strain of human cytomegalovirus (HCMV), repaired for the expression of a functional UL130 protein (Towneᵣₑₚ).

For infection, cells from 8 flasks were combined and collected by low speed centrifugation (150×g, 5 min., room temperature). After that, cells were resuspended in 2×32 mL of CAP-T Express medium, containing Towneᵣₑₚ in a concentration to result in a m.o.i. of 1 or 5. For each m.o.i., 4 mL of infected cells were transferred to each of a total of 8 flasks which were shaken at 50 rpm for 4 hours in the incubator. Following that 16 mL of CAP-T Express medium was supplemented to each flask and the flasks were shaken at 200 rpm.

After 4 and 6 days, respectively, the culture supernatants were collected. For this, low speed centrifugation was performed to remove the cells (150×g, 5 min., room temperature). Supernatants of four flasks were combined and centrifuged (1300×g, 10 Min., room temperature). One mL of the supernatant was then saved for further analysis of the virus titer and was stored at −80° C.

The remaining supernatant was then centrifuged at 100,000×g (70 Min., 10° C.) in a SW32Ti rotor in a Beckman Optima L-90K ultracentrifuge. Meanwhile, the gradients were prepared by mixing 4 mL 35% sodium-tartrate solution with 5 mL 15% sodium-tartrate/30% Glycerin-solution in 0.04 M sodium-phosphate buffer pH 7.4, using a gradient mixer and Beckman Ultra-Clear™ centrifuge tubes (14×89 mm). Following centrifugation, the pellets were resuspended in 700 μl 1×PBS. For measure of the protein concentration in these samples, 15 μl were removed and stored at −80° C. until further analysis. The rest of the samples were applied on top of one gradient. Centrifugation was performed at 91,000×g (60 min., 10° C.) in a SW41 rotor.

After centrifugation, the bands, corresponding to NIEPs, virions and DB were visualized by light scattering and collected from the gradient, using a syringe and a 80 G×1.5″-gauge needle.

Each sample was supplemented with 1×PBS to give a total volume of 10 mL. Centrifugation was then performed at 99,000×g (90 min., 10° C.) in a SW41 rotor. The pellets were resuspended in 50 μl (virions) or 100 μl (NIEPs, DB) 1×PBS. 15 μl were taken for further analyses of protein concentration and stored at −80° C. until further use. The residual samples were stored in aliquots.

The protein concentrations in the samples were evaluated by using the Pierce® BCA Protein Assay Kit (Thermo Scientific, Order-No.: 23225) according to the manufacturer's instructions.

TABLE 1
Summary of the different analyses performed for viral particles
release from infected CAP cells in suspension:

| Infection # | 2A | 2B | 2C | 2D |
|---|---|---|---|---|
| Virus | RV- | RV- | RV- | RV- |
| | TowneUL130 | TowneUL130 | TowneUL130 | TowneUL130 |
| | repaired | repaired | repaired | repaired |
| Number of cells per vial | 5 × 10⁶/4 | 5 × 10⁶/4 | 5 × 10⁶/4 | 5 × 10⁶/4 |
| (20 ml medium)/ | | | | |
| Number of vials | | | | |
| m.o.i. | 1 | 1 | 5 | 5 |
| Day of virus purification | 4 | 6 | 4 | 6 |
| (p.i.) | | | | |
| Total protein (μg) | 1032 | 1470 | 1421 | 2355 |
| preceding gradient | | | | |
| purification | | | | |
| Protein content (μg) in | 130 | 101 | 307 | 234 |
| the “NIEPs” fraction | | | | |
| Protein content (μg) in | 3 | not | 9 | 5 |
| the “virions” fraction | | collected | | |
| Protein content (μg) in | 44 | 405 | 35 | 214 |
| the “dense body” fraction | | | | |

Abbreviation: p.i.: post infection

In order to further evaluate the proteins in the NIEPS and DB fractions released from suspension CAP cells infected with different 1 or 5 m.o.i. a 10% SDS-polyacrylamide gel was used for the separation of the proteins in the samples.

Separation gels: 10 mL distilled water, 6.25 mL Tris 1.5 M (pH 8.8), 8.3 mL Gel 30 (Rotiphorese® Gel 30, Roth) 250 μL 10% SDS, 250 μL 10% APS, 15 μL TEMED.

Stacking gels: 4.30 mL distilled water, 0.75 mL Tris 1 M (pH 6.8), 1.05 mL Gel 30 (Rotiphorese® Gel 30, Roth), 0.62 μL 10% SDS, 0.62 μL 10% APS, 7.5 μL TEMED.

After polymerisation, gels were mounted on a vertical gel chamber (Hoefer SE 600 Series Electrophoresis Unit; U.S. Pat. No. 4,224,134). The running buffer (upper and lower buffer chamber) contained 1×PAGE buffer (200 mL 5×PAGE-Puffer [25 mM Tris-Base, 192 mM glycine, 0.1% SDS]+800 mL distilled water). 2 or 10 μL pre-stained protein marker (PeqGOLD Protein Marker IV from Peqlab; cat. no. 27-2110) were applied. Either 2 μg (FIG. 14 A) or 5 μg (FIG. 14 B) total proteins were separated overnight at 4.35 V/cm (50 V, 400 mA and 100 W; Electrophoresis Power Supply—EPS 600; Pharmacia Biotech). The glass plates were removed the following day and the gels were rinsed in water.

For the staining of the proteins, the glass plates were removed the following day and the gels were rinsed in water. Silver staining was done using the Roti®-Black P-silver staining kit for proteins (Roth, order-No. L533.1) according to the manufacturer's instructions.

The analyses presented in FIG. 14 showed that CAP cells release particles that correspond in their sedimentation properties to the NIEPs, DB and virions released from HFF. NIEPs and DB appeared to band in prominent fractions whereas virions were purified in low amounts, only. Surprisingly, the results show that high amounts of Dense Bodies were released from the HCMV infected cells.

### EXAMPLE 15

### Evaluation of DB Production in Suspension CAP Cells by Immunoblot Analysis

For the immunoblot analysis of the DB fractions obtained from suspension CAP cells proteins were separated on a polyacrylamide gel as described in example 14.

For immunoblot analysis by Odyssey, the PVDF filter (PVDF-Membrane: “Millipore” Immobilon-FL, Cat. No. IPFL00010) was cut in appropriate pieces and was rinsed in methanol for 5 minutes, briefly rinsed in water and then equilibrated for 20 minutes in transfer buffer/10% vol/vol methanol (25 mM Tris, 192 mM glycine, 10% methanol, ad 2 L distilled water). The gel was also incubated in transfer buffer for 20 minutes. The initial layer on the semi-dry blot apparatus (Hölzel) consisted of three layers of chromatography paper (Chromatography Paper Whatman®) which was pre-soaked in transfer buffer. Next, the equilibrated PVDF-membrane was applied, avoiding the generation of air-bubbles. After that the gel was applied, again avoiding air-bubbles. The final stack consisted of 3 layers of chromatography paper. The transfer was performed using 600V, 400 mA for 75 minutes. After that, the apparatus was dismantled and the PVDF-membrane with the transferred proteins was air-dried for 1 to 2 hours in a fume hood. The membrane was then briefly rinsed in methanol, followed by rinsing it in water. After an incubation in 1×PBS for a few minutes, blocking reagent (5% w/v dry milk powder [Roth, Art. Nr. T145.2] in 1×PBS) was applied and the filters were incubated on a tumbling device for 1 hour.

The primary murine monoclonal antibody 65-33, directed against pp65 (ppUL83) of HCMV (obtained from Prof. W. Britt, UAB, Birmingham, Ala., USA) was diluted 1:2,000 in blocking reagent/0.1% v/v Tween100. Membranes were incubated with the primary antibody in film wraps over night at room temperature, avoiding trapping for air bubbles. The next day, the antibody was removed and the filters were washed three times for 10 minutes in 1×PBS/0.2% v/v Tween100. After that, secondary antibodies were applied (IRDye 800 conjugated affinity purified goat-anti-mouse-IgG, Rockland, Order-No. 610-132-121), in a dilution of 1:5.000 in blocking reagent/0.1% v/v Tween100/0.01% v/v SDS. Incubation was performed in the dark for two hours at room temperature. After that, the secondary antibody was removed by 2 washing steps for 10 minutes in 1×PBS/0.2% Tween100 and 1 washing step for 10 minutes in 1×PBS in the dark. Evaluation of the results was performed with an “Odyssey Imager” (LI-COR; Lincoln, Nebr., USA).

For immunoblot analysis by ECL-staining, the PVDF filter (PVDF-Membrane: “Millipore” Immobilon-PSQ, Cat. No. ISEQ00010) was cut in appropriate pieces and was rinsed in methanol for 5 minutes and then equilibrated for 20 minutes in transfer buffer/10% vol/vol methanol (25 mM Tris, 192 mM glycine, 10% methanol, ad 2 L distilled water). The gel was also incubated in transfer buffer for 20 minutes. The initial layer on the semi-dry blot apparatus (Hölzel) consisted of three layers of chromatography paper (Chromatography Paper Whatman®) which was pre-soaked in transfer buffer. Next, the equilibrated PVDF-membrane was applied, avoiding the generation of air-bubbles. After that the gel was applied, again avoiding air-bubbles. The final stack consisted of 3 layers of chromatography paper. The transfer was performed using 2 mA/cm² for 90 minutes.

After that, the apparatus was dismantled and the PVDF membrane was incubated on a tumbling device for 2 hours in blocking reagent (5% w/v dry milk powder [Roth, Art. Nr. T145.2] in 1×PBS). The primary murine monoclonal antibody 65-33, directed against pp65 (ppUL83) of HCMV (obtained from Prof. W. Britt, UAB, Birmingham, Ala., USA) was diluted 1:2,000 in 5% w/v blocking reagent/0.1% v/v Tween100 in 1×PBS.

The primary murine monoclonal antibody gB (27-287), directed against gB of HCMV (obtained from Prof. W. Britt, UAB, Birmingham, Ala., USA) was diluted 1:2 in 10% w/v blocking reagent/0.1% v/v Tween100 in 1×PBS.

The primary polyclonal pp65-specific rabbit antiserum 65R, directed against pp65 (ppUL83) of HCMV was diluted 1:10000 in 5% w/v blocking reagent/0.1% v/v Tween100 in 1×PBS. The primary polyclonal DB-specific rabbit antiserum, directed against DB of HCMV was diluted 1:5000 in 5% w/v blocking reagent/0.1% v/v Tween100 in 1×PBS.

Membranes were incubated with the primary antibody in film wraps over night at +4° C., avoiding trapping for air bubbles. The next day, the antibody was removed and the filters were washed four times for 20 minutes in 1×PBS/0.1% v/v Tween100. After that, secondary antibodies were applied (rabbit-anti-mouse-HRP, Dako, order-No. P0260 against monoclonal antibodies and swine-anti-rabbit-HRP Dako, order-No. P0217 against polyclonal antibodies), in a dilution of 1:10,000 in blocking reagent/0.1% v/v Tween100 and the membranes incubated in film wraps for one hour at room temperature avoiding trapping for air bubbles. After incubation, the filters were washed four times for 20 minutes in 1×PBS/0.1% v/v Tween100 and after that for 15 minutes in 1×PBS. Antigen-detection was done by using the Amersham ECL Plus Western Blotting Detection Reagents-Kit (GE Healthcare, order-No. RPN2132) according to the manufacturer's instructions.

The DB fraction displayed the expected high abundance of pp65 and gB protein in immunoblot analysis.

The results presented in FIGS. 14, 15 and table 1 demonstrate that CAP cells, grown in suspension in serum-free medium, release particles that correspond in their sedimentaion properties to NIEPs, DB and virions produced in HFF. NIEPs and DBs appeared in prominent fractions. Thus, these results demonstrate that suspension CAP cells are appropriate for DB production.

### EXAMPLE 16

### Analysis of Morphological Changes and Viral Protein Expression in Suspension CAP Cells Infected with HCMV

To analyze the morphological changes displayed by HCMV infected suspension CAP cells and to investigate the expression of IE1 and pp65, cytospin slides of infected cells were prepared. For this, on day 1, 2 and 3 after infection 1×10⁴-1×10⁵ cells were harvested and centrifuged at 150×g for 5 minutes, respectively. Cells were subsequently resuspended in 100 μL 1×PBS and transferred into Schandon Cytoslides (coated; 76×26×1 mm; Thermo Scientific; 5991056). Centrifugation was performed in a Cytospin 4 cytocentrifuge (Thermo Scientific) at 150×g for 5 minutes. The slides were subsequently allowed to air-dry for 30 minutes. After that the cells were fixed using an equal mixture of acetone and methanol. Slides were allowed to air-dry for at least 30 minutes.

To investigate expression of particular viral proteins, the slides were stained with specific antibodies, using the alkaline-phosphatase-anti-alkaline-phosphatase (APAAP) technology.

For staining of pp65, the Clonab CMV antibody from Biotest (Dreieich, Germany) was used in a dilution of 1:10 in 1×TBS, with 1% bovine serum albumin (BSA). 25 μL of that dilution was applied to each cytospin spot. Incubation was for 30 minutes at room temperature in a wet chamber. For staining of IE1, the monoclonal antibody p63-27 (Hybridoma cell supernatant, donation of Prof. W. Britt, University of Alabama at Birmingham, Birmingham, Ala. USA) was used undiluted and proceeded as described above. After incubation, slides were washed two times in 1×TBS for 1 to 5 minutes, each.

In a second step, bridging antibody anti-Maus-Ig (Sigma M 5899 or Dako Z 0259) was applied at a dilution of 1:25 in TBS with 1% BSA. 25 μL of that solution was applied to each spot. Incubation was for 30 minutes at room temperature in a wet chamber. After incubation, slides were washed two times in 1×TBS for 1 to 5 minutes, each.

In a next step, the antibody from the alkaline-phosphatase-anti-alkaline-phosphatase system from Sigma (A 7827) or Dako (D 0651) was applied. The antibody was diluted 1:50 in 1×TBS with 1% BSA. 25 μL of that solution was applied to each spot. Incubation was for 30 minutes at room temperature in a wet chamber. After incubation, slides were washed two times in 1×TBS for 1 to 5 minutes, each.

For staining, 1 drop of Fuchsine+chromogen and 1 drop of Fuchsine+activating agent (Fuchsin+Substrate-Chromogen System, Dako Code K 625) were mixed. 700 μL of substrate buffer were added. The substrate solution was directly applied at 50 μL in each spot. Incubation was for 10 to 12 min in a wet chamber. Following that, the slides were briefly rinsed three times in TBS and briefly with distilled water.

Following that slides were incubated in filtrated hematoxilin (Harris, Gill 2, Merck) for 1 minute. After that, slides were incubated in tap water for 3 to 5 minutes. Spots were covered with 10 to 12 mm cover slips using mounting medium or an equal mixture of glycerol and distilled water. Cells were then inspected using an Axiophot microscope (Zeiss).

The results presented in FIGS. 17 and 18 showed that over 90% of the CAP cells were initially infected, as represented by the IE1-specific nuclear staining at day 1 post infection. Also at day 1 post infection cells showed pp65 staining, yet at a lower frequency. At day 2 and, more pronounced at day 3 post infection, most cells had lost the IE1 staining. The number of cells showing pp65 expression increased over time and at day 3 post infection, most of the pp65 signal was cytoplasmic. The results show that the nucleophilic tegument protein pp65 was translocated to the cytoplasm of the cells, a process considered necessary for Dense Body morphogenesis.

The cells displayed a marked cytopathic effect, mostly at day 2 and 3 post infection or at later phases of infection. The morphology of the cells was such that both nuclear and plasma membrane appeared to be disrupted thereby releasing most of the internal structures. Taken together the experiments showed that penetration of HCMV into CAP cells was highly efficient and that the cells were permissive for the initial events of viral IE (intermediate early)- and E (early)-gene expression.

### EXAMPLE 17

### Production of Permanent Human Amniocyte Cell Lines (Adherent CAP Cells)

### 1. Cloning Procedures

The production of permanent human amniocyte cell lines is exemplary described in EP 1 230 354 B1. In the following three exemplary plasmids are described which can be used for the transfection of primary amniocyte cells.

a) Plasmid pSTK146

Plasmid pSTK146 was described in detail in EP 1 230 354 B1 and comprises the murine phosphoglycerate kinase (pgk) promoter, adenovirus serotype 5 (Ad5) sequences nucleotide (nt.) 505 to 3522 and the splicing and polyadenylation signal of SV40.

b) Plasmid pGS119

Plasmid pGS119 contains the murine pgk promoter, Ad5 sequences nt. 505-3522 containing the entire E1 region, the 3′ splicing and polyadenylation signal of SV40 and the pIX region of Ad5 (nt. 3485-4079).

The Ad5 pIX gene sequences are derived from plasmid pXCl (Microbix Biosystems Inc, Catalogue No. PD-01-03) containing Ad5 sequences nt. 22-5790. Using this plasmid and the primer p9.3485-3504 (CTGGCTCGAGCTCTAGCGATGAAGATACAG; SEQ ID NO:1) and p9.4079-4060 (GCTGCTCGAGCACTTGCTTGATCCAAATCC; SEQ ID NO:2) Ad5 gene sequences nt. 3485-4079 were amplified by polymerase chain reaction (PCR), cleaved with XhoI (each primer contains one XhoI restriction site) and introduced into the XhoI restriction site of pSTK146.

c) Plasmid pGS122

Plasmid pGS122 contains Ad5 sequences by 1-4344. In a first step, Ad5 sequences nt. 356-3826 were isolated from pXCl (Microbix Biosystems Inc, Catalogue No. PD-01-03) using SacII digestion and introduced into the SacII restriction site of pSTK31 (contains a PmeI restriction site followed by Ad5 sequences by 1-400 in pBluescript). The in such a way generated plasmid pGS120 was linearised with BstEII, and the BstEII fragment from pXCl containing the Ad5 sequences by 1914-5185 was introduced (pGS121). Two oligonucleotides Ad5_{—}4297-4344.PX (GCTGGGCGTGGTGCCTAAAAATGTCTTTCAGTAGCAAGCTGA TTGCCAGTTTAAAC; SEQ ID NO:3) and Ad5_{—}4344-4297 (TCGAGTTTAAACTGGCAA TCAGCTTGCTACTGAAAGACATTTTTAGGCACCACGCCCAGCT; SEQ ID NO:4) were hybridised to form the double strand. Plasmid pGS121 was digested with AfeI and XhoI, and the above-mentioned oligonucleotide was introduced. The sequence of the oligonucleotide was selected such that upon introduction into pGS121 the AfeI restriction site at the 5′ end is retained, and at the 3′ end a PmeI restriction site is followed by the regenerated XhoI restriction site. Thus, the Ad5 sequences in pGS122 are directly flanked by a PmeI restriction site.

### 2. Verification of the Constructs

### a) Sequence Analysis

The completeness of all plasmids described above was tested by restriction digest. Furthermore, the correct sequence of the adenovirus fragments in pSTK146, pGS119 and pGS122 was confirmed by sequence analysis; no alterations in the sequence as compared to the Ad5 wildtype sequence were found.

### b) Expression

The plasmids pSTK146, pGS119 and pGS122 were transfected into HeLa cells and the expression of the E1A proteins was analysed by Western blotting using a monoclonal antibody (see chapter 6).

### 3. Cultivation of Cells

### a) Cell Lines

The cell culture reagents were obtained, unless indicated differently, from the company Invitrogen GmbH. HEK293 and HeLa cells were cultivated in modified in Eagle's Medium (MEM) with 10% foetal calf serum (FCS), 1× penicillin/streptomycin at 37° C., 95% humidity and 5% CO₂.

### b) Primary Amniocytes

Primary amniocytes were, following routine methods, obtained during an amniocentesis. 1-2 ml of this amniotic puncture were cultivated with 5 ml Ham's F10 medium, 10% FCS, 2% Ultroser G (CytoGen GmbH), 1× antibiotic/antimycotic at 37° C., 95% humidity and 5% CO₂ in 6 cm-Primaria cell culture dishes (Falcon). After 4-6 days the amniocytes started to become adherent, and 3 ml fresh medium plus additives (see above) were added. As soon as the cells were fully adherent, the medium was removed and replaced by 5 ml fresh medium plus additives. For the further passages the confluent cells were washed with PBS, detached with trypsin (TrypleSelect, Invitrogen) and transferred into 10 and 25 ml, respectively, fresh medium plus additives into 10 cm and 15 cm dishes, respectively.

### 4. Transfection and Transformation of Primary Amniocytes

The primary amniocytes were transfected by the transfection of the above described plasmids. For the transfection all plasmids except pGS122 were linearised prior to the transfection by a digest with suitable restriction nucleases. The plasmid pGS122 was digested with PmeI prior to the transfection since the adenovirus sequences in pGS122 are flanked by one PmeI restriction site each. For the transfection 2 μg plasmid were used. Transformed cell clones could be obtained with all plasmids and single clones could be isolated and tested.

Hereinafter, the generation of CAP cells using the plasmid pGS119 is described in detail exemplary.

Prior to the transfection, the amniocytes were adapted to Opti-Pro medium with 2% Ultroser. For this purpose, the cells were spiked with fresh Ham's F10 medium (with additives) plus Opti-Pro medium (with 2% Ultroser) in a ratio of 75:25%, 50:50%, 25:75% and 0:100% every 2-3 days.

For the transfection, the cells of an approximately 80% confluent 15 cm dish were distributed onto 6 cm dishes corresponding to a cell number of 5-7×10⁵ cells per dish. On the following day, the cells on 5 dishes were transfected with 2 μg pGS119, linearised with ScaI, using the transfection reagent Effectene (Qiagen) according to the manufacturer's protocol. One dish was not transfected and cultivated as a control. On the next day, the cells were washed with PBS, detached with TrypleSelect and transferred to a 15 cm dish. The cells were cultivated for further 10-15 days, wherein the medium was replaced by fresh medium every 3-4 days. During this time the addition of Ultroser was decreased to 1%. After about 10-15 days the cells were confluent and were transferred to 15 cm dishes, as described above.

### 5. Isolation of the Transformed Cell Clones

A few weeks after the transfection, clonal cell islands could be observed, which were significantly morphologically distinct from the non-transformed amniocytes. These cell islands were picked and transferred onto 24-well-dishes (corresponding to passage 1). The cells were furthermore propagated and transferred to 6 cm dishes at first and later to 15 cm dishes.

Initially, about 40 clones were isolated, which partially were significantly morphologically distinct from each other during the further cultivation. Some of these clones showed a significant “crisis” in the case of a prolonged cultivation, i.e., they were very instable in their growth. The further experiments were limited to the further cultivation and analysis of seven morphologically stable cell clones.

### 6. Characterisation of the Cell Lines—Expression of the E1 Genes (Western Blot)

The expression of the E1A and E1B 21 kD proteins was detected in the seven clonal cell lines by Western blot analysis using monoclonal antibodies.

For this purpose, 7×10⁵ cells of each of the individual cell clones were plated in a 6-well-dish each. Seventy-two hours later the cells were detached with Tris-saline/4 mM EDTA, pelleted, resuspended in 100 μl Tris-saline and lysed by the addition of 30 μl 4×SDS-loading buffer (40% glycerol, 1.4 M mercaptoethanol, 8% SDS, 250 mM Tris/HCl pH 7). As a negative control a lysate was used which was prepared from the same number of primary amniocytes. As a positive control served a lysate of HEK293 cells. 10 μl each of these protein mixtures were electrophoretically separated on a 10% SDS polyacrylamide gel and transferred onto a nitrocellulose membrane. The membrane bound proteins were visualised by chemoluminescence (ECL, Amersham) by the incubation with an anti-E1A and anti-E1B 21 kD antibody, respectively, (Oncogene Research) and an HRP-conjugated anti-mouse (E1A, Jackson ImmunoResearch Laboratories) and an anti-rat (E1B 21 kD, Oncogene Research) antibody, respectively, and subsequent incubation. The results of the Western blots with E1A and E1B demonstrated that all examined cell lines express the E1A proteins (3 bands at 30-50 kD) and the E1B 21 kD protein.

1. A method for the production of human Cytomegalovirus (HCMV) particles comprising the steps of:
(a) contacting and thereby infecting a permanent human amniocyte cell with HCMV,
(b) incubating the amniocyte cell to facilitate expression of HCMV particles, and
(c) isolating the HCMV particles,
wherein the permanent human amniocyte cell expresses the adenoviral gene products E1A and E1B, and wherein the amniocyte cells in step (a) are cultured in serum-free medium.

2. The method according to claim 1, wherein the amniocyte cells are adherent cells or suspension cells.

3. The method according to claim 1, wherein the HCMV particles in step (c) are isolated from the medium or from the intracellular space of the amniocyte cell.

4. The method according to claim 1, wherein the isolating in step (c) is conducted from the medium with rate velocity gradient centrifugation, density gradient differential centrifugation or zone centrifugation.

5. The method according to claim 1, wherein the permanent human amniocyte cell is grown in the lag phase, the exponential phase or the stationary phase during the time of the contacting and infecting with HCMV in step (a).

6. The method according to claim 1, wherein the adenoviral gene products E1A and E1B comprise the nucleotide 1 to 4344, 505 to 3522 or nucleotide 505 to 4079 of the human adenovirus serotype-5.

7. The method according to claim 1, wherein the permanent human amniocyte cell further expresses the adenoviral the adenoviral gene product pIX.

8. The method according to claim 1, wherein the amniocyte cell is contacted and infected in step (a) with HCMV in an amount in the range of 0.001 to 10 MOI.

9. Human Cytomegalovirus (HCMV particles produced according to a method according to claim 1.

10. HCMV particles according to claim 9, wherein said HCMV particles comprise Dense Bodies.

11. HCMV particles according to claim 10, wherein said HCMV particles comprise a fraction of Dense Bodies of 10 to 90% in relation to the total protein amount of the fractions of NIEPs, virions and Dense Bodies.

12. A human Cytomegalovirus (HCMV)-based vaccine comprising HCMV particles according to claim 9.

13.

[symbol]

14. A method of preparing a human Cytomegalovirus (HCMV)-based therapeutic or diagnostic agent comprising placing HCMV particles according to claim 9 into a pharmaceutically acceptable composition.

15. A method for the prevention or treatment of human Cytomegalovirus (HCMV) related disease comprising administering to a subject in need thereof the HCMV vaccine of claim 12.

16. A method for the diagnosis of a human Cytomegalovirus (HCMV) infection comprising contacting an antibody-containing patient sample with HCMV particles of claim 9.
